(19) United States
(12) Patent Application Publication
(10) Pub. No.: US 2023/0130754 A1
(43) Pub. Date: Apr. 27, 2023

(54) SURFACE-MODIFIED EXOSOMES AND METHODS OF USE

(71) Applicant: The Board of Regents of the University of Oklahoma, Norman, OK (US)

(72) Inventors: Dongin Kim, Norman, OK (US); Chang Sun KANG, Oklahoma City, OK (US); Sun Young LEE, Oklahoma City, OK (US)

(21) Appl. No.: 18/050,195

(22) Filed: Oct. 27, 2022

Related U.S. Application Data
(60) Provisional application No. 63/272,424, filed on Oct. 27, 2021.

(51) Int. Cl. C07K 16/22; A61P 35/00; C07K 17/04

(52) U.S. Cl. CPC C07K 16/22; A61P 35/00; C07K 17/04; C07K 2319/035

(57) ABSTRACT

Modified exosomes are disclosed that include an exosome and a targeting modality that extends outwardly from a surface membrane of the exosome. Also disclosed are methods of producing and using the modified exosomes.

### CROSS-REFERENCE TO RELATED APPLICATIONS

This application claims the benefit of U.S. Provisional Application No. 63/272,424, filed Oct. 27, 2021, the disclosure of which is expressly incorporated herein by reference in its entirety.

### BACKGROUND

The site-specific targeting and delivery of therapeutics and/or diagnostic agents has been a long-sought goal in biology and medicine. The integrin αᵥβ₃, for example, is overexpressed in primary melanoma cancer cells, thus is a cancer-specific receptor that can be targeted through known interactions between RGD peptide and αᵥβ₃ integrin. Certain research has characterized the therapeutic and diagnostic potentials of RGD-mediated targeting of αᵥβ₃ on cancer cells. For example, RGD-coated lipid nanoparticles have shown significant inhibition of αᵥβ₃-overexpressing breast cancer cells. Paclitaxel (PTX) and iron oxide encapsulated nanoparticles coated with RGD peptide not only inhibited the growth of αᵥβ₃ expressing tumors but also enabled visualization of the tumor.

With the discoveries of the roles of exosomes as cell-cell communication mediators, cellular regulators, and RNA and protein carriers, the uses of exosomes in therapies against various diseases have been investigated. Exosomes, which are naturally-derived liposomes, have gained attention as a potent platform in the field of drug delivery system due to their intrinsic cell-homing ability, and superior biocompatibility in comparison to synthetic nanoparticles. However, various design parameters, including therapeutic cargos, loading methods, administration routes, dose, and use of targeting moiety, must be satisfied for exosome-based drug delivery platforms to achieve therapeutic success. A key challenge to using exosomes as therapeutics is the requirement of high doses due to their short systemic half-life (e.g., <10 minutes). Non-specific accumulation of systemically-administered exosomes exacerbated by limited research in embedding active-targeting functions into exosomes has limited their potential as therapeutics. Efforts have been made to modify exosomes to enable target-specific activity. Currently, two primary ways of decorating exosome surfaces with targeting ligands include chemical conjugation, and genetic transfection of parent cells with ligand inducing genes. However, chemical conjugation not only prevents control over the amount of bound ligands due to unpredictable variations in surface densities of functional groups but can also disrupt the integrity of the exosome structure and function during synthesis. Currently, few studies have identified membrane anchoring as a viable strategy for exosome surface modification, primarily using cholesterols or peptides or lipid components. While these materials successfully served as anchors within exosome membranes, they have not demonstrated capacity to significantly broaden the repertoire of existing exosome functions. Genetic transfection of parent cells requires long culture times and faces challenges in reproducibly expressing ligand on exosomes.

Thus, an efficient, simple, safe, and controllable method for generating active targeting exosomes is still needed. It is to addressing this need that certain embodiments of the present disclosure are directed.

Intravitreal injection of various medications including vascular endothelial growth factor (VEGF) neutralizing proteins, steroid or antibiotics is mainstay for treating many of posterior eye diseases. Employment of intravitreal injections opened up the new therapeutic revenue in intraocular drug delivery in the last decade. Intravitreal injection provides improved efficacy of drug delivery than systemic admiration to treat posterior eye diseases because of numbers of reasons, such as overcoming anatomical blood-retinal barriers or benefits of intraocular local drug delivery minimizing treatment related systemic side effects. Especially, the number of intravitreal injections of anti-VEGF agents has grown exponentially with the introduction of Bevacizumab (Avastin®), Ranibizumab (Lucentis®), and most recently Aflibercept (Eylea®) to treat ocular neovascularization in several retinal and choroidal vascular diseases including neovascular age-related macular degeneration (NVAMD), diabetic retinopathy (DR), macular edema, and retinal vein occlusion (RVO). Although millions of intravitreal injections are performed each year in the United States with indisputable vision improving benefit, many patients still experience suboptimal visual outcomes due to insufficient treatment efficacy. The suboptimal efficacy of anti-VEGF monotherapy may be due to several factors. Local delivery of intravitreally injected anti-VEGF agents to the retina primarily depends on diffusion from vitreous humor to retina and choroid without active targeting of NV lesions. Biological impedance within vitreoretinal interface-retina-retina pigment epithelium (RPE) tight junction-choroid is a major barrier to efficacy. Frequently, the accompanying retinal fibrosis and gliosis secondary to NV are additional barriers to efficient penetration of intravitreally delivered drugs, and currently there is no treatment for retinal fibrosis. In addition, direct injection of drugs relies on monotherapy due to limited allowed volume for intravitreal injection at any given time. Accordingly, anti-VEGF monotherapy, mostly commonly used intravitreal injection treatment, does not target multiple signaling pathways involved in ocular NV. Further, clearance of intravitreally delivered anti-VEGF agents through the aqueous and vitreous humor shortens the half-life of these treatments (2-4 days). Hence, socioeconomic burden of frequently required long-term monthly injections to maintain therapeutic efficacy of the anti-VEGF agents is a practical barrier to optimal efficacy. Therefore, new approaches to the current anti-VEGF monotherapy that can provide active targeting of NV, have the capacity to deliver multiple drugs, and maintain long-term efficacy are necessary.

### BRIEF DESCRIPTION OF THE DRAWINGS

This patent or application file contains at least one drawing executed in color. Copies of this patent or patent application publication with color drawings will be provided by the Office upon request and payment of the necessary fee.

FIG. 1 is a schematic diagram showing how, in accordance with the present disclosure, exosomes can be functionalized and used as a theranostic agent for cancer therapy, for example.

FIG. 2 shows the development of an anchor-spacer-ligand conjugate (ASL) as a fluorometric decoration tool to exosomes (a) A synthetic route of ASL. (b)¹H NMR spectrum of ASL recorded in D₂O. (c) UV-vis absorption and (d) fluorescence spectra of dEx and dAEx in PBS, and ASL, BODIPY, and DOX were dissolved in DMSO.

FIG. 3 shows an ¹H NMR spectrum of compound 1 recorded in D₂O.

FIG. 4 shows results of the molecular weight (MW) distribution of ASL through MALDI-TOF analysis.

FIG. 5 shows an elution profile of ASL using a SEPHACRYL S100 column.

FIG. 6 shows results characterizing ASL-decorated exosomes. (a) Size distribution of doxorubicin-encapsulated AExs (dAEx) as determined by DLS. (b) Representative transmission electron micrograph of dAEx. (c) Size distribution profile of dAEx generated by NTA. (d) Elution profile of dAEx from SEPHACRYL S100 column. (e) Release kinetics of ASL and DOX from dAEx, and BODIPY from BODIPY-labeled Ex. (f) Expression of exosomes-associated protein after generation of dAEx. Improved stability of AEx. (g) The change in the hydrodynamic size (h) Concentration of particles in the presence of FBS (10%). (i) Binding activity of AEx to integrin αᵥβ₃. Values are mean±SD(n=3, ***p<0.01 and ****p<0.001).

FIG. 7 shows results characterizing free exosomes (Ex). (a) Size distribution determined by dynamic light scattering. (b) Representative transmission electron micrograph. (c) Size distribution profile generated by nanoparticle tracking analysis.

FIG. 8 shows the Zeta potential distribution of (a) Ex and (b) dAEx.

FIG. 9 shows the Integrin αᵥβ₃-dependent or non-specific binding of ASL.

FIG. 10 demonstrates the enhanced anticancer activity of dAEx. (a) Comparison of cytotoxicity of ASL and AEx against RAW264.7 cells. (b) Relative cell viability against B16F10 cells after treating dAEx, dEx, DOX and RGD+dAEx at different concentrations. (c) In vitro cytotoxicity of various agents at 3 μm of DOX determined by CCK-8 assay. Expression of (d) cleaved caspase-3 and (e) cleaved PARP of B16F10 cells. (f) Western blot analysis of apoptosis-related proteins of B16F10 cells. Values are mean±SD (n=3. **p<0.05, ***p<0.01, ****p<0.001).

FIG. 11 shows a comparison of the cytotoxicity of ASL and AEx against B16F10 cells.

FIG. 12 shows the expression of integrin αᵥβ₃ of b16F10 cells.

FIG. 13 shows results of the cellular uptake of dAEx in B16F10 cells incubated with dEx and dAEx for 3 h. (a) Confocal microscopy images. Scale bar represents to 30 μm. Intracellular mean fluorescence intensity of (b) DOX and (c) ASL. Values are mean±SD (n=9˜28, and ****p<0.001).

FIG. 14 shows the therapeutic anticancer activity of dAEx. Changes of (a) tumor volumes, (b) body weight. (c) Images of mice bearing B16F10 tumor xenograft with various formulations and plotted tumor weight of mice. values are mean±SD (n=3˜6, **p<0.05, ***p<0.01, and ****p<0.001).

FIG. 15 shows the tumor growth in individual animals after treatment. (a) Untreated, (b) dAEx, (c) dEx, (d) DOX, (e), ASL, and (f) Ex.

FIG. 16 shows a histological analysis of tumor tissues. (a) Representative H&E-stained images of tumor tissues. Scale bar, 100 μm. (b) Representative TUNEL stained images of tumor tissues. Scale bar represents to 200 μm.

FIG. 17 shows results of the tumor-targeting activity of dAEx. (a) Representative fluorescence image of tumor bearing mouse after administration of dAEx. Time-dependent biodistribution and pharmacokinetics of tumor and organs. (b) Tumor accumulation as a function of time. Fluorescence signals of (c) ASL from dAEx-treated mice, and (d) BODIPY from mice treated with BODIPY-labeled Ex.

FIG. 18 shows histology analysis of in vivo toxicity of AEx using H&E-stained organs from AEx-treated mice (X200). Scale bar represent to 100 μm.

FIG. 19 shows retinal uptake of intravitreally delivered exosomes shown in fluorescence tracing images. C57BL/6J mice received intravitreal injection of Müller glia-derived PKH26-labeled (red, white arrows) or mouse retina derived ASL-modified exosomes (green, yellow arrows). (A and B) In vivo retinal imaging microscopy followed by intraperitoneal injection of FITC-dextran which labels blood vessels (green) under green channel where exosomes transported to the retina see at 3 and 24 hours after exosome treatment. (C and D) Confocal microscopy of retinal sections show exosome penetration to the retina. Exosomes were taken at IPL, INL and 1 day after intravitreal injection. (E) Intravitreally injected ASL-exosomes (green, yellow arrows) showed similar pattern of retinal uptake in healthy wild type retina, shown in 3D reconstructed images from retina flat mount 1 day after intravitreal injection using Fiji and associated plugins.

FIG. 20 shows increased binding activity of ASL-exosome (AEx) to integrin αᵥβ₃. ASL fluorescence was measured from integrin-immobilized wells. Values are mean±SD. **** p<0.001 for difference from multiple groups by ANOVA.

FIG. 21 shows results from a laser-induced choroidal neovascularization (CNV) mouse model and measurement of the total volume of CNV. Laser induced mouse model of CNV was confirmed 7 days after laser by (A) fundus photography, (B) FA, (C) OCT, and (D) H&E staining from retinal section (8 μm). Choroid/RPE were dissected and stained with rhodamine-conjugated GSA (E). 3 D image of total volume of CNV was reconstructed and measured using Fiji and associated plugins (F).

FIG. 22 shows that ASL-exosomes actively target CNV, colocalized with increased expression of integrin αᵥ at the site of CNV. Intravitreal injection of wild type mouse retina-derived ASL-exosomes (1 μl) was given 3 days after laser treatment in a mouse model of CNV and eyes were harvested 7 days after injection. In retinal sections, ASL-exosomes (green) were predominantly taken to the area of CNV, compared with no exosome uptake in non-lasered areas. ASL-exosomes (green) were colocalized with increasingly expressed integrin αᵥ (red), of 89.5% colocalization within CNV (A and B). In RPE/choroid flat mounts stained with GSA (green), integrin αᵥ (red) was increasingly expressed (C) and ASL-exosomes (green, white arrows) predominantly taken to the area of CNV were co-localized with integrin αᵥ (D). Some of ASL-exosomes locally delivered to CNV were observed intracellularly (E). * Exosome particles are only visible in X40 due to nano size.

FIG. 23 shows in vivo intracellular uptake of ASL-exosomes by vascular endothelial cells and macrophages at CNV sites. Retinal sections obtained from experiment in FIG. 26 were stained with DAPI and antibodies of anti-ICAM1 (red) and anti-F4/80 (red). Intracellular uptake of ASL-exosomes (green, white arrows) by both vascular endothelial cells and macrophages were observed.

FIG. 24 shows that intravitreal exosome treatment did not induce reactive retinal gliosis. C57BL/6J mice received intravitreal injection of PKH26-labeled exosomes (red, white arrows). Retinal section was obtained 1 day after exosome injection and was stained with GFAP (green), a sensitive marker for reactive retinal gliosis. Exosomes were taken at IPL, INL and OPL at 1 day and GFAP expression was not increased around the exosomes. GFAP is constitutively expressed in GCL in normal and non-stressed retina. N=3, scale bar 50 μm.

FIG. 25 shows that Eylea-loaded ASL-exosomes (Ex-ASL-Exo) treatment improved CNV suppression by 20% compared with Eylea alone treatment. Laser-induced CNV mouse model on C57BL/6 mice were intravitreally injected with 1 μl of Ex-ASL-Exo, Eylea, naive exosomes, or PBS 3 days after laser treatment. After 7 days of exosome treatments, choroid/RPE flat mounts were obtained and stained with rhodamine-conjugated GSA. Total volume of CNV was measured using Fiji and associated plugins. Bars represent mean (±SEM). * p<0.005, *** p<0.0001 for difference from multiple groups by ANOVA with Kruskal-Wallis and Dunn's test. Scale bar, 50 μm. N=10-12 per group.

FIG. 26 shows a scheme of the disclosed process. ASL-exosomes have targeted binding to ocular NV lesions by actively binding to increasingly expressed extracellular integrins in NV and by increased intracellular uptake of exosomes through integrin receptor-mediated intracellular endocytosis. ASL-exosomes can co-deliver Eylea and miR-24 and provide effective and sustained suppression of NV.

### DETAILED DESCRIPTION

The present disclosure is directed to, in at least certain embodiments, an innovative exosome surface-modification system utilizing intramembrane incorporation of a synthetic anchor-spacer-ligand (ASL) conjugate, forming an ASL-incorporated exosome (AEx). The anchor molecule is incorporated into the exosome membrane enabling the targeting ligand to extend from the outer surface of the exosome. In an exemplary, non-limiting embodiment, the anchor can be a fluorescent lipophilic boron-dipyrromethene (BODIPY), which can incorporate into the exosome membrane due to its hydrophobicity/lipophilicity. Since BODIPY can be used as a biodistribution and pharmacokinetic marker because of its fluorescence intensity, it also enables the detection and measurement of the insertion and retention kinetics of the ASL conjugate within exosomes. The intermediate spacer portion of the ASL helps to extend the half-life of the exosome in circulation by preventing exosome fusion and aggregation at higher concentrations and minimizing systemic clearance by the reticuloendothelial system (RES). In a non-limiting embodiment, a poly(ethylene glycol) (PEG) molecule can be used as the spacer between the anchor and targeting ligand molecules and an Arg-Gly-Asp (RGD) peptide can be used as a targeting ligand, e.g., for targeting an integrin overexpressed on cancer cells (e.g., melanoma and ovarian carcinoma) of in neovascularized areas of the eye (e.g., in the retina or choroid). ASLs not only give stability but also provide cell-specific targetability and imaging capabilities enabling exosomes to be used as theranostic agents. ASL-engineered exosomes enhance the therapeutic efficacies of therapeutic drugs. In a mouse model of cancer, drug-loaded AEx achieved tumor-targeted imaging, and significantly suppressed the tumor development without distinct side effects. In a mouse model of ocular NV, AEx loaded with an anti-NV agent significantly reduced NV in the eye. ASLs can thus be utilized with exosomes to enable both therapeutic and diagnostic applications.

Before further describing various embodiments of the present disclosure in more detail by way of exemplary description, examples, and results, it is to be understood that the compounds, compositions, agents, and methods of present disclosure are not limited in application to the details of specific embodiments and examples as set forth in the following description. The description provided herein is intended for purposes of illustration only and is not intended to be construed in a limiting sense. As such, the language used herein is intended to be given the broadest possible scope and meaning, and the embodiments and examples are meant to be exemplary, not exhaustive. Also, it is to be understood that the phraseology and terminology employed herein is for the purpose of description only and should not be regarded as limiting unless otherwise indicated as so. Moreover, in the following detailed description, numerous specific details are set forth in order to provide a more thorough understanding of the present disclosure. However, it will be apparent to a person having ordinary skill in the art that the present disclosure may be practiced without these specific details. In other instances, features which are well known to persons of ordinary skill in the art have not been described in detail to avoid unnecessary complication of the description. It is intended that all alternatives, substitutions, modifications, and equivalents apparent to those having ordinary skill in the art are included within the scope of the present disclosure. All of the compounds, compositions, agents, and methods and applications and uses thereof disclosed herein can be made and executed without undue experimentation in light of the present disclosure. Thus, while the compounds, compositions, agents, and methods of the present disclosure have been described in terms of particular embodiments, it will be apparent to those of skill in the art that variations may be applied to the compounds, compositions, agents, and methods and in the steps or in the sequence of steps of the methods described herein without departing from the concept, spirit, and scope of the inventive concepts.

All patents, patent application publications, and non-patent publications, including published articles, which are identified, listed, or mentioned in the specification or referenced in any portion of this application are herein expressly incorporated by reference in their entirety to the same extent as if each individual patent, patent application publication, and non-patent publication was specifically and individually indicated to be incorporated by reference.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular. Where used herein, the specific term “single” is limited to only “one.”

As utilized in accordance with the methods, compounds, agents, and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, includes ranges of 1-20, 10-50, 50-100, 100-500, and 500-1,000, for example. Reference to an integer with more (greater) or less than includes any number greater or less than the reference number, respectively. Thus, for example, reference to less than 100 includes 99, 98, 97, etc. all the way down to the number one (1); and less than 10 includes 9, 8, 7, etc. all the way down to the number one (1).

As used in this specification and claims, the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” and “approximately” used to indicate that a value includes the inherent variation of error for the composition, the method used to administer the composition, or the variation that exists among the study subjects. “About” or “approximately” are intended to include not only the exact value, amount, degree, orientation, or other qualified characteristic or value, but are intended to include some slight variations due to measuring error, manufacturing tolerances, stress exerted on various parts or components, observer error, wear and tear, and combinations thereof, for example. The term “about” or “approximately,” where used herein when referring to a measurable value such as an amount, a temporal duration, and the like, is meant to encompass, for example, variations of ±20% or ±10%, or ±5%, or ±1%, or ±0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

As used herein any reference to “one embodiment” or “an embodiment” means that a particular element, feature, structure, or characteristic described in connection with the embodiment is included in at least one embodiment and may be included in other embodiments. The appearances of the phrase “in one embodiment” in various places in the specification are not necessarily all referring to the same embodiment and are not necessarily limited to a single or particular embodiment. Further, all references to one or more embodiments or examples are to be construed as non-limiting to the claims.

The term “pharmaceutically acceptable” refers to compounds, agents, and compositions which are suitable for administration to humans and/or animals without undue adverse side effects such as toxicity, irritation and/or allergic response commensurate with a reasonable benefit/risk ratio. The compounds or conjugates of the present disclosure may be combined with one or more pharmaceutically-acceptable excipients, including carriers, vehicles, and diluents which may improve solubility, deliverability, dispersion, stability, and/or conformational integrity of the compounds or conjugates thereof.

By “biologically active” is meant the ability to modify the physiological system of an organism without reference to how the active agent has its physiological effects.

As used herein, “pure” or “substantially pure” means an object species is the predominant species present (i.e., on a molar basis it is more abundant than any other object species in the composition thereof), and particularly a substantially purified fraction is a composition wherein the object species comprises at least about 50 percent (on a molar basis) of all macromolecular species present. Generally, a substantially pure composition will comprise more than about 80% of all macromolecular species present in the composition, more particularly more than about 85%, more than about 90%, more than about 95%, or more than about 99%. The term “pure” or “substantially pure” also refers to preparations where the object species is at least 60% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure.

The term “subject” is used interchangeably herein with the term “patient” and includes human and veterinary subjects. For purposes of treatment, the term “mammal” as used herein refers to any animal classified as a mammal, including (but not limited to) humans, non-human primates, Old and New World monkeys, domestic animals (such as, but not limited to, dogs and cats), experimental mammals (such as mice, rats, rabbits, guinea pigs, and chinchillas), farm animals (such as, but not limited to, horses, pigs, cattle, goats, sheep, and llamas), zoo animals, and any other animal that has mammary tissue.

“Treatment” refers to therapeutic treatments. “Prevention” refers to prophylactic or preventative treatment measures or reducing the onset of a condition or disease. The term “treating” refers to administering the composition to a subject for therapeutic purposes and/or for prevention. The terms “treat,” “treating” and “treatment,” as used herein, will be understood to include both inhibition of cancerous cell growth or bacterial or parasite growth as well as killing parasites and/or infected cells.

The term “receptor” as used herein will be understood to include any peptide, protein, glycoprotein, lipoprotein, polycarbohydrate, or lipid that is expressed or overexpressed on the surface of a cell.

The terms “therapeutic composition” and “pharmaceutical composition” refer to an active agent-containing composition that may be administered to a subject by any method known in the art or otherwise contemplated herein, wherein administration of the composition brings about a therapeutic effect as described elsewhere herein. In addition, the compositions of the present disclosure may be designed to provide delayed, controlled, extended, and/or sustained release using formulation techniques which are well known in the art. An active agent is a compound or other treatment and/or diagnostic modality (e.g., an ASL-exosome) which has a therapeutic and/or diagnostic benefit in accordance with the present disclosure. The term “theranostic” refers to an active agent which has both a therapeutic and a diagnostic activity in accordance with the present disclosure.

The term “effective amount” refers to an amount of an active agent which is sufficient to exhibit a detectable therapeutic, diagnostic, or treatment effect in a subject without excessive adverse side effects (such as substantial toxicity, irritation, and allergic response) commensurate with a reasonable benefit/risk ratio when used in the manner of the present disclosure. The effective amount for a subject will depend upon the subject's type, size, and health, the nature and severity of the condition to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

The term “ameliorate” means a detectable or measurable improvement in a subject's condition, disease, or symptom thereof. A detectable or measurable improvement includes a subjective or objective decrease, reduction, inhibition, suppression, limit, or control in the occurrence, frequency, severity, progression, or duration of the condition or disease, or an improvement in a symptom or an underlying cause or a consequence of the disease, or a reversal of the disease. A successful treatment outcome can lead to a “therapeutic effect” or “benefit” of ameliorating, decreasing, reducing, inhibiting, suppressing, limiting, controlling, or preventing the occurrence, frequency, severity, progression, or duration of a disease or condition, or consequences of the disease or condition in a subject.

A decrease or reduction in worsening, such as stabilizing the condition or disease, is also a successful treatment outcome. A therapeutic benefit therefore need not be complete ablation or reversal of the disease or condition, or any one, most, or all adverse symptoms, complications, consequences, or underlying causes associated with the disease or condition. Thus, a satisfactory endpoint may be achieved when there is an incremental improvement such as a partial decrease, reduction, inhibition, suppression, limit, control, or prevention in the occurrence, frequency, severity, progression, or duration, or inhibition or reversal of the condition or disease (e.g., stabilizing), over a short or long duration of time (hours, days, weeks, months, etc.). Effectiveness of a method or use, such as a treatment that provides a potential therapeutic benefit or improvement of a condition or disease, can be ascertained by various methods and testing assays.

As used herein, the term “concurrent therapy” is used interchangeably with the terms “combination therapy” and “adjunct therapy,” and will be understood to mean that the patient in need of treatment is treated or given another drug for the disease in conjunction with the conjugated exosomes of the present disclosure. This concurrent therapy can be sequential therapy where the patient is treated first with one drug and then the other, or the two drugs are given simultaneously. In certain embodiments, the subject may be administered, with the ASL-exosome, an additional therapeutic and/or diagnostic agent. The additional agent may be administered simultaneously, within the same or different compositions, or may be administered sequentially. For example, the AEx may be administered first and the additional agent administered second. Or the AEx may be administered after the additional agent is administered.

The term “exosome” as used herein, refers to a membranous particle having a diameter (or largest dimension where the particles is not spheroid) of between about 10 nm to about 5000 nm, more typically between 30 nm and 1000 nm, and most typically between about 50 nm and 750 nm, wherein at least part of the membrane of the exosome is directly obtained from a donor cell. Most commonly, exosomes will have a size (average diameter) that is up to 5% of the size of the donor cell. Therefore, especially contemplated exosomes include those that are shed from a cell.

Exosomes may be detected in or isolated from any suitable sample type, such as, for example, body fluids. As used herein, the term “isolated” refers to separation out of its natural environment and is meant to include at least partial purification and may include substantial purification. As used herein, the term “sample” refers to any sample suitable for the methods provided by the present invention. The sample may be any sample that includes exosomes suitable for detection or isolation. Sources of samples include blood, bone marrow, pleural fluid, peritoneal fluid, cerebrospinal fluid, urine, saliva, amniotic fluid, malignant ascites, broncho-alveolar lavage fluid, synovial fluid, breast milk, sweat, tears, joint fluid, and bronchial washes. In one aspect, the sample is a blood sample, including, for example, whole blood or any fraction or component thereof. A blood sample suitable for use with the present invention may be extracted from any source known that includes blood cells or components thereof, such as venous, arterial, peripheral, tissue, cord, and the like. For example, a sample may be obtained and processed using well-known and routine clinical methods (e.g., procedures for drawing and processing whole blood). In one aspect, an exemplary sample may be peripheral blood drawn from a subject with cancer.

Exosomes may also be isolated from tissue samples, such as surgical samples, biopsy samples, tissues, feces, and cultured cells. When isolating exosomes from tissue sources it may be necessary to homogenize the tissue in order to obtain a single cell suspension followed by lysis of the cells to release the exosomes. When isolating exosomes from tissue samples it is important to select homogenization and lysis procedures that do not result in disruption of the exosomes. Exosomes contemplated herein are preferably isolated from body fluid in a physiologically acceptable solution, for example, buffered saline, growth medium, various aqueous medium, etc. Exosomes may be isolated from freshly collected samples or from samples that have been stored frozen or refrigerated. In some embodiments, exosomes may be isolated from cell culture medium. Although not necessary, higher purity exosomes may be obtained if fluid samples are clarified before precipitation with a volume-excluding polymer, to remove any debris from the sample. Methods of clarification include centrifugation, ultracentrifugation, filtration, or ultrafiltration. Most typically, exosomes can be isolated by numerous methods well-known in the art. One method is differential centrifugation from body fluids or cell culture supernatants.

One accepted protocol for isolation of exosomes includes ultracentrifugation, often in combination with sucrose density gradients or sucrose cushions to float the relatively low-density exosomes. Isolation of exosomes by sequential differential centrifugations is complicated by the possibility of overlapping size distributions with other microvesicles or macromolecular complexes. Furthermore, centrifugation may provide insufficient means to separate vesicles based on their sizes. However, sequential centrifugations, when combined with sucrose gradient ultracentrifugation, can provide high enrichment of exosomes.

In non-limiting embodiments, the ligands of the ASLs may be antibodies or portions thereof. The term “antibody” as used herein can refer to both intact, “full length” antibodies as well as to fragments thereof which are able to bind to a desired target site on a cell. Such antibody fragments may also be referred to herein as antigen binding fragments, antigen binding portions, binding fragments, or binding portions thereof. As used herein, the term “antibody” includes, but is not limited to, synthetic antibodies, monoclonal antibodies, recombinantly produced antibodies, intrabodies, multispecific antibodies (including bi-specific antibodies), human antibodies, humanized antibodies, chimeric antibodies, recombinant single chain polypeptide molecules in which light and heavy chain variable regions are connected by a peptide linker, i.e., single-chain Fv (scFv) fragments, bivalent scFv (bi-scFv), trivalent scFv (tri-scFv), Fab fragments, Fab′ fragments, F(ab′) fragments, F(ab′)2 fragments, F(ab)₂ fragments, disulfide-linked Fvs (sdFv) (including bi-specific sdFvs), and anti-idiotypic (anti-Id) antibodies, diabodies, dAb fragments, nanobodies, diabodies, triabodies, tetrabodies, linear antibodies, isolated CDRs, and epitope-binding fragments of any of the above. Regardless of structure, an antibody fragment binds with the same antigen that is recognized by the intact antibody. Fragments can be produced by recombinant DNA techniques or by enzymatic or chemical separation of intact immunoglobulins.

The antibodies of several embodiments provided herein may be monospecific, bispecific, trispecific, or of greater multispecificity, such as multispecific antibodies formed from antibody fragments. The term “antibody” also includes a diabody (homodimeric Fv fragment) or a minibody (V_{L}-V_{H}-C_{H3}), a bispecific antibody, or the like. A bispecific or bifunctional antibody is an artificial hybrid antibody having two different heavy/light chain pairs and two different binding sites. Multispecific antibodies may be specific for different epitopes of a polypeptide or may be specific for both a polypeptide as well as for a heterologous epitope, such as a heterologous polypeptide or solid support material. Single chain antibodies produced by joining antibody fragments using recombinant methods, or a synthetic linker, are also encompassed by the present disclosure (e.g., see, for example, International Patent Application Publication Nos. WO 93/17715; WO 92/08802; WO 91/00360; and WO 92/05793; and U.S. Pat. Nos. 4,474,893; 4,714,681; 4,925,648; 5,573,920; and 5,601,819).

The compositions, formulations, and methods described herein may include monoclonal antibodies. Rodent monoclonal antibodies to specific antigens may be obtained by methods known to those skilled in the art (e.g., see Kohler and Milstein, op.cit., and Coligan et al. (eds.), Current Protocols in Immunology, Vol. 1, pages 2.5.1-2.6.7 (John Wiley & Sons 1991)). General techniques for cloning murine immunoglobulin variable domains have been disclosed, for example, by the publication of Orlandi et al. (Proc. Nat'l Acad. Sci. USA, 86: 3833 (1989)). The term “monoclonal antibody” as used herein refers to an antibody obtained from a population of substantially homogeneous antibodies, i.e., the individual antibodies comprising the population are identical except for possible naturally occurring mutations that may be present in minor amounts. The modifier “monoclonal” indicates the character of the antibody as being obtained from a substantially homogeneous population of antibodies, and is not to be construed as requiring production of the antibody by any particular method. For example, the monoclonal antibodies used in accordance with the present disclosure can be made by the hybridoma method first described by Kohler et al. (Nature, 256:495 (1975)), or may be made by recombinant DNA methods (see, for example, U.S. Pat. No. 4,816,567).

An “isolated” antibody refers to an antibody that has been identified and separated and/or recovered from components of its natural environment and/or an antibody that is recombinantly produced. A “purified antibody” is an antibody that is typically at least 50% w/w pure of interfering proteins and other contaminants arising from its production or purification but does not exclude the possibility that the monoclonal antibody is combined with an excess of pharmaceutical acceptable carrier(s) or other vehicle(s) intended to facilitate its use. Interfering proteins and other contaminants can include, for example, cellular components of the cells from which an antibody is isolated or recombinantly produced. Sometimes monoclonal antibodies are at least 60%, at least 70%, at least 80%, at least 90%, at least 95%, or at least 99% w/w pure of interfering proteins and contaminants from production or purification. The antibodies described herein, including murine, chimeric, and humanized antibodies, can be provided in isolated and/or purified form.

A “therapeutic agent” is an atom, molecule, radiation, or compound that is useful in the treatment of a disease. Examples of therapeutic agents include but are not limited to antibodies, antibody fragments, drugs, cytokine or chemokine inhibitors, pro-apoptotic agents, tyrosine kinase inhibitors, toxins, enzymes, nucleases, hormones, immunomodulators, antisense oligonucleotides, siRNA, RNAi, chelators, boron compounds, photoactive agents, dyes, radiation, and radioisotopes.

A “diagnostic agent” is an atom, molecule, or compound that is useful in diagnosing a disease. Useful diagnostic agents include, but are not limited to, radiation, radioisotopes, dyes, contrast agents, fluorescent compounds or molecules, and enhancing agents (e.g., paramagnetic ions). In certain particular (but non-limiting) embodiments, the diagnostic agents are selected from the group comprising radioisotopes, enhancing agents, and fluorescent compounds.

As used herein, the term “antibody fusion protein” is a recombinantly produced antigen-binding molecule in which an antibody or antibody fragment is linked to another protein or peptide, such as the same or different antibody or antibody fragment. The fusion protein may comprise a single antibody component, a multivalent or multispecific combination of different antibody components, or multiple copies of the same antibody component. The fusion protein may additionally comprise an antibody or an antibody fragment and a therapeutic agent.

For purposes of classifying amino acids substitutions as conservative or nonconservative, amino acids are grouped in one non-limiting embodiment as follows: Group I (hydrophobic side chains): met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same group. Nonconservative substitutions constitute exchanging a member of one of these groups for a member of another.

Tables of conservative amino acid substitutions have been constructed and are known in the art. In other embodiments, examples of interchangeable amino acids include, but are not limited to, the following: arginine and lysine; glutamate and aspartate; serine and threonine; glutamine and asparagine; and valine, leucine, and isoleucine. In other non-limiting embodiments, the following substitutions can be made: Ala (A) by leu, ile, or val; Arg (R) by gln, asn, or lys; Asn (N) by his, asp, lys, arg, or gln; Asp (D) by asn or glu; Cys (C) by ala or ser; Gln (Q) by glu or asn; Glu (E) by gln or asp; Gly (G) by ala; His (H) by asn, gln, lys, or arg; Ile (I) by val, met, ala, phe, or leu; Leu (L) by val, met, ala, phe, or ile; Lys (K) by gln, asn, or arg; Met (M) by phe, ile, or leu; Phe (F) by leu, val, ile, ala, or tyr; Pro (P) by ala; Ser (S) by thr; Thr (T) by ser; Trp (W) by phe or tyr; Tyr (Y) by trp, phe, thr, or ser; and Val (V) by ile, leu, met, phe, or ala.

Other considerations for amino acid substitutions include whether or not the residue is located in the interior of a protein or is solvent—(i.e., externally) exposed. For interior residues, conservative substitutions include for example: Asp and Asn; Ser and Thr; Ser and Ala; Thr and Ala; Ala and Gly; Ile and Val; Val and Leu; Leu and Ile; Leu and Met; Phe and Tyr; and Tyr and Trp. For solvent-exposed residues, conservative substitutions include for example: Asp and Asn; Asp and Glu; Glu and Gln; Glu and Ala; Gly and Asn; Ala and Pro; Ala and Gly; Ala and Ser; Ala and Lys; Ser and Thr; Lys and Arg; Val and Leu; Leu and Ile; Ile and Val; and Phe and Tyr.

Percentage sequence identities can be determined with antibody sequences maximally aligned by the Kabat numbering convention. After alignment, if a particular antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage.

Compositions or methods “comprising” one or more recited elements may include other elements not specifically recited. For example, a composition that comprises an antibody may contain the antibody alone or in combination with other ingredients.

The phrase “pharmaceutically acceptable salt” refers to pharmaceutically acceptable organic or inorganic salts of a therapeutic molecule. Exemplary salts include sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as (but not limited to) an acetate ion, a succinate ion, or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterions.

Where used herein the term “annexin” refers to any of annexins 1-11 and 13, which are more particularly designated as annexins A1, A2, A3, A4, A5, A6, A7, A8, A9, A10, All, and A13. Annexin I and annexin V, where used herein, refer to Annexin A1 and Annexin A5, respectively, for example. The annexins contemplated herein further include non-human cognate orthologs of A1-All and A13 from non-human vertebrates, including but not limited to, non-human primates, dogs, cats, horses, livestock animals and zoo animals, which may be used for treatment in said non-human mammals in the methods contemplated herein. The annexins contemplated for use herein are discussed in further detail in V. Gerke and S. E. Moss (Physiol. Rev., 82:331-371 (2002)), the entirety of which is expressly incorporated by reference herein.

A humanized antibody is a genetically engineered antibody in which the variable heavy and variable light CDRs from a non-human “donor” antibody are grafted into human “acceptor” antibody sequences (see for example, U.S. Pat. Nos. 5,530,101; 5,585,089; 5,225,539; 6,407,213; 5,859,205; and 6,881,557). The acceptor antibody sequences can be, for example, a mature human antibody sequence, a composite of such sequences, a consensus sequence of human antibody sequences, or a germline region sequence. Thus, a humanized antibody is an antibody having some or all CDRs entirely or substantially from a non-human donor antibody and variable region framework sequences and constant regions, if present, entirely or substantially from human antibody sequences. Similarly, a humanized heavy chain has at least one, two, and usually all three CDRs entirely or substantially from a donor antibody heavy chain, and a heavy chain variable region framework sequence, and heavy chain constant region, if present, substantially from human heavy chain variable region framework and constant region sequences. Similarly, a humanized light chain has at least one, two, and usually all three CDRs entirely or substantially from a donor antibody light chain, and a light chain variable region framework sequence and light chain constant region, if present, substantially from human light chain variable region framework and constant region sequences. Other than nanobodies and dAbs, a humanized antibody comprises a humanized heavy chain and a humanized light chain.

The AEx used in the present compositions and methods of treatment can be formulated into compositions for delivery to a mammalian subject. The composition can be administered alone and/or mixed with a pharmaceutically acceptable vehicle or excipient. Suitable vehicles are, for example (but not by way of limitation), water, saline, dextrose, glycerol, ethanol, or the like, and combinations thereof. In addition, the vehicle can contain minor amounts of auxiliary substances such as (but not limited to) wetting or emulsifying agents, pH buffering agents, or adjuvants. The compositions of the present disclosure can also include ancillary substances, such as (but not limited to) pharmacological agents, cytokines, or other biological response modifiers.

Furthermore, the compositions can be formulated into compositions in either neutral or salt forms. Pharmaceutically acceptable salts include (but are not limited to) the acid addition salts (formed with the free amino groups of the active polypeptides) and which are formed with inorganic acids such as, for example, hydrochloric or phosphoric acids, or organic acids such as acetic, oxalic, tartaric, mandelic, and the like. Salts formed from free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, and procaine.

Compositions can be administered in a single dose treatment or in multiple dose treatments on a schedule and over a time period appropriate to the age, weight, and condition of the subject, the particular composition used, and the route of administration. In one non-limiting embodiment, a single dose of the composition according to the disclosure is administered. In other non-limiting embodiments, multiple doses are administered. The frequency of administration can vary depending on any of a variety of factors, e.g., severity of the symptoms, or whether the composition is used for prophylactic or curative purposes. For example, in certain non-limiting embodiments, the composition is administered once per month, twice per month, three times per month, every other week, once per week, twice per week, three times per week, four times per week, five times per week, six times per week, every other day, daily, twice a day, or three times a day. The duration of treatment (i.e., the period of time over which the composition is administered) can vary, depending on any of a variety of factors, e.g., subject response. For example, the composition can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

The compositions can be combined with a pharmaceutically acceptable carrier (excipient) to form a pharmacological composition. Pharmaceutically acceptable carriers can contain a physiologically acceptable compound that acts to, for example but not by way of limitation) stabilize or increase or decrease the absorption or clearance rates of the pharmaceutical compositions. Physiologically acceptable compounds can include, for example but not by way of limitation: carbohydrates, such as glucose, sucrose, or dextrans; antioxidants, such as ascorbic acid or glutathione; chelating agents; low molecular weight proteins; detergents; liposomal carriers; excipients; or other stabilizers and/or buffers. Other physiologically acceptable compounds include (but are not limited to) wetting agents, emulsifying agents, dispersing agents, or preservatives.

When administered orally, the present compositions may be protected from digestion. This can be accomplished either by combining the AEx with a composition to render it resistant to acidic and enzymatic hydrolysis or by packaging the AEx in an appropriately resistant carrier such as (but not limited to) a liposome, e.g., such as shown in U.S. Pat. No. 5,391,377.

For transmucosal or transdermal administration, penetrants appropriate to the barrier to be permeated can be used in the formulation. Such penetrants are generally known in the art, and include, e.g., for transmucosal administration, bile salts and fusidic acid derivatives. In addition, detergents can be used to facilitate permeation. Transmucosal administration can be through nasal sprays or using suppositories. For topical transdermal administration, the agents are formulated into ointments, creams, salves, powders, and gels. Transdermal delivery systems can also include (for example but not by way of limitation) patches. The present compositions can also be administered in sustained delivery or sustained release mechanisms. For example, biodegradable microspheres or capsules or other biodegradable polymer configurations capable of sustained delivery of a peptide can be included herein.

For inhalation, the present compositions can be delivered using any system known in the art, including (but not limited to) dry powder aerosols, liquids delivery systems, air jet nebulizers, propellant systems, and the like. For example (but not by way of limitation), the pharmaceutical formulation can be administered in the form of an aerosol or mist. For aerosol administration, the formulation can be supplied in finely divided form along with a surfactant and propellant. In another aspect, the device for delivering the formulation to respiratory tissue is an inhaler in which the formulation vaporizes. Other liquid delivery systems include (for example but not by way of limitation) air jet nebulizers.

The AEx can be delivered alone or as pharmaceutical compositions by any means known in the art, such as (but not limited to) systemically, regionally, or locally; by intra-arterial, intrathecal (IT), intravenous (IV), parenteral, intra-pleural cavity, topical, oral, or local administration, as subcutaneous, intra-tracheal (e.g., by aerosol) or transmucosal (e.g., buccal, bladder, vaginal, uterine, rectal, nasal mucosa).

In one aspect, the compositions are prepared with carriers that will protect the AEx against rapid elimination from the body, such as (but not limited to) a controlled release formulation, including implants and microencapsulated delivery systems. Biodegradable, biocompatible polymers can be used, such as (but not limited to) ethylene vinyl acetate, polyanhydrides, polyglycolic acid, collagen, polyorthoesters, and polylactic acid. Methods for preparation of such formulations will be apparent to those skilled in the art.

The AEx in general may be formulated to obtain compositions that include one or more pharmaceutically suitable excipients, surfactants, polyols, buffers, salts, amino acids, or additional ingredients, or some combination of these. This can be accomplished by known methods to prepare pharmaceutically useful dosages, whereby the active compound is combined in a mixture with one or more pharmaceutically suitable excipients. Sterile phosphate-buffered saline is one non-limiting example of a pharmaceutically suitable excipient.

Non-limiting examples of routes of administration of the compositions described herein include parenteral injection, e.g., by subcutaneous, intramuscular, or transdermal delivery. Other forms of parenteral administration include (but are not limited to) intravenous, intraarterial, intralymphatic, intrathecal, intraocular (e.g., intravitreal, subconjunctival, subretinal, or sub-Tenon's injection), intracerebral, or intracavitary injection. In parenteral administration, the compositions will be formulated in a unit dosage injectable form such as (but not limited to) a solution, suspension, or emulsion, in association with a pharmaceutically acceptable excipient. Such excipients are inherently nontoxic and nontherapeutic. Non-limiting examples of such excipients include saline, Ringer's solution, dextrose solution, and Hanks' solution. Nonaqueous excipients such as (but not limited to) fixed oils and ethyl oleate may also be used. An alternative non-limiting excipient is 5% dextrose in saline. The excipient may contain minor amounts of additives such as (but not limited to) substances that enhance isotonicity and chemical stability, including buffers and preservatives.

Formulated compositions comprising the AEx can be used (for example but not by way of limitation) for subcutaneous, intramuscular, or transdermal administration. Compositions can be presented in unit dosage form, e.g., in ampoules or in multi-dose containers, with an added preservative. Compositions can also take such forms as suspensions, solutions, or emulsions in oily or aqueous vehicles, and can contain formulatory agents such as suspending, stabilizing, and/or dispersing agents. Alternatively, the compositions can be in powder form for constitution with a suitable vehicle, e.g., sterile pyrogen-free water, before use.

The compositions may be administered in solution. The formulation thereof may be in a solution having a suitable pharmaceutically acceptable buffer, such as (but not limited to) phosphate, Tris (hydroxymethyl) aminomethane-HCl, or citrate, and the like. Buffer concentrations should be in the range of 1 to 100 mM. The formulated solution may also contain a salt, such as (but not limited to) sodium chloride or potassium chloride in a concentration of 50 to 150 mM. An effective amount of a stabilizing agent such as (but not limited to) mannitol, trehalose, sorbitol, glycerol, albumin, a globulin, a detergent, a gelatin, a protamine, or a salt of protamine may also be included.

Exemplary, non-limiting ranges for a therapeutically or prophylactically effective amount of an AEx, include a range of from about 0.001 mg/kg of the subject's body weight to about 500 mg/kg of the subject's body weight, such as but not limited to a range of from about 0.01 mg/kg to about 250 mg/kg, a range of from about 0.1 mg/kg to about 100 mg/kg, a range of from about 0.1 mg/kg to about 50 mg/kg, a range of from about 1 mg/kg to about 30 mg/kg, a range of from about 1 mg/kg to about 25 mg/kg, a range of from about 2 mg/kg to about 30 mg/kg, a range of from about 2 mg/kg to about 20 mg/kg, a range of from about 2 mg/kg to about 15 mg/kg, a range of from about 2 mg/kg to about 12 mg/kg, a range of from about 2 mg/kg to about 10 mg/kg, a range of from about 3 mg/kg to about 30 mg/kg, a range of from about 3 mg/kg to about 20 mg/kg, a range of from about 3 mg/kg to about 15 mg/kg, a range of from about 3 mg/kg to about 12 mg/kg, or a range of from about 3 mg/kg to about 10 mg/kg, or a range of from about 10 mg to about 1500 mg as a fixed dosage.

The composition is formulated to contain an effective amount of the AEx, wherein the amount depends on the subject to be treated and the severity of the condition of the subject. In certain non-limiting embodiments, the present AEx may be administered at a dose ranging from about 0.001 mg to about 10 g, from about 0.01 mg to about 10 g, from about 0.1 mg to about 10 g, from about 1 mg to about 10 g, from about 1 mg to about 9 g, from about 1 mg to about 8 g, from about 1 mg to about 7 g, from about 1 mg to about 6 g, from about 1 mg to about 5 g, from about 10 mg to about 10 g, from about 50 mg to about 5 g, from about 50 mg to about 5 g, from about 50 mg to about 2 g, from about 0.05 μg to about 1.5 mg, from about 10 μg to about 1 mg protein, from about 30 μg to about 500 μg, from about 40 μg to about 300 μg, from about 0.1 μg to about 200 mg, from about 0.1 μg to about 5 μg, from about 5 μg to about 10 μg, from about 10 μg to about 25 μg, from about 25 μg to about 50 μg, from about 50 μg to about 100 μg, from about 100 μg to about 500 μg, from about 500 μg to about 1 mg, or from about 1 mg to about 2 mg. The specific dose level for any particular subject depends upon a variety of factors, including (but not limited to) the activity of the specific AEx, the age, body weight, general health, sex, diet, time of administration, route of administration, and rate of excretion, the drug combination, and the severity of the disease in the subject undergoing therapy.

In certain embodiments of ocular treatments, the active agent or a salt thereof is administered locally to, into, in or around the eye in a dose from about 10-500 μg, 50-500 μg or 100-500 μg per administration (e.g., by eye drop or injection). In certain embodiments, the statin is administered locally in a dose from about 10-50 μg, 50-100 μg, 100-200 μg, 200-300 μg, 300-400 μg or 400-500 μg per administration (e.g., by eye drop or injection). In other embodiments, the active agent is administered locally in a dose from about 10 or 20 μg to about 200 μg, or from about 10 or 20 μg to about 100 μg, per administration. In further embodiments, the active agent or a salt thereof is administered locally to, into, in or around the eye in a total or cumulative dose of about 0.1 or 0.3-15 mg or 0.5 or 1-10 mg over a period of about 1 month. In certain embodiments, the active agent is administered locally (e.g., by eye drop, injection or implant) in a total dose of about 0.1 or 0.3-1 mg, 1-5 mg, 5-10 mg or 10-15 mg over a period of about 1 month. In other embodiments, the active agent is administered locally (e.g., by eye drop, injection or implant) in a total dose of about 0.5-10 mg or 0.5-5 mg over a period of about 1 month. In still further embodiments, the active agent or a salt thereof is administered locally to, into, in or around the eye (e.g., by eye drop, injection or implant) in a total or cumulative dose of about 0.5 or 2-100 mg, 5 or 10-100 mg, or 5 or 10-50 mg over a period of about 6 months. In certain embodiments, the active agent is administered locally (e.g., by eye drop, injection or implant) in a total dose of about 0.5-2 mg, 2-10 mg, 0.5-5 mg, 5-10 mg, 10-50 mg or 50-100 mg over a period of about 6 months. In other embodiments, the active agent is administered locally (e.g., by eye drop, injection or implant) in a total dose from about 2 or 5 mg to about 50 mg, or from about 2 or 5 mg to about 25 mg, over a period of about 6 months. In additional embodiments, the active agent or a salt thereof is administered locally to, into, in or around the eye (e.g., by eye drop, injection or implant) in a total or cumulative dose of about 1 or 4-200 mg, 5 or 10-200 mg, 5 or 10-150 mg, or 5 or 10-100 mg for the whole or entire treatment regimen. In certain embodiments, the active agent is administered locally (e.g., by eye drop, injection or implant) in a total dose of about 1-5 mg, 5-10 mg, 1-10 mg, 10-50 mg, 50-100 mg, 100-150 mg or 150-200 mg for the entire treatment regimen. In other embodiments, the active agent is administered locally (e.g., by eye drop, injection or implant) in a total dose from about 5 or 10 mg to about 100 mg, or from about 5 or 10 mg to about 50 mg, for the entire treatment regimen.

The dosage of an administered AEx for humans will vary depending upon factors such as (but not limited to) the patient's age, weight, height, sex, general medical condition, and previous medical history. In certain non-limiting embodiments, the recipient is provided with a dosage of the AEx (s) that is in the range of from about 1 mg to about 1000 mg as a single infusion or single or multiple injections, although a lower or higher dosage also may be administered. In certain non-limiting embodiments, the dosage may be in the range of from about 25 mg to about 100 mg per square meter (m²) of body surface area for a typical adult, although a lower or higher dosage also may be administered. Non-limiting examples of dosages that may be administered to a human subject further include 1 to 500 mg, 1 to 70 mg, or 1 to 20 mg, although higher or lower doses may be used. Dosages may be repeated as needed, for example (but not by way of limitation), once per week for 4-10 weeks, once per week for 8 weeks, or once per week for 4 weeks. It may also be given less frequently, such as (but not limited to) every other week for several months, or more frequently, such as twice weekly or by continuous infusion.

In some non-limiting embodiments, the amount of an AEx is in a concentration of about 1 nM, about 5 nM, about 10 nM, about 25 nM, about 50 nM, about 75 nM, about 100 nM, about 150 nM, about 200 nM, about 250 nM, about 300 nM, about 350 nM, about 400 nM, about 500 nM, about 550 nM, about 600 nM, about 700 nM, about 800 nM, about 900 nM, about 1 μM, about 2 μM, about 3 μM, about 4 μM, about 5 μM, about 6 μM, about 7 μM, about 8 μM, about 9 μM, about 10 μM, about 15 μM, about 20 μM, about 25 μM, about 30 μM, about 35 μM, about 40 μM, about 45 μM, about 50 μM, about 60 μM, about 70 μM, about 75 μM, about 80 μM, about 90 μM, about 100 μM, about 125 μM, about 150 μM, about 175 μM, about 200 μM, about 250 μM, about 300 μM, about 350 μM, about 400 μM, about 500 μM, about 600 μM, about 700 μM, about 750 μM, about 800 μM, about 900 μM, about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 100 mM, about 110 mM, about 120 mM, about 130 mM, about 140 mM, about 150 mM, about 160 mM, about 170 mM, about 180 mM, about 190 mM, about 200 mM, about 250 mM, about 300 mM, about 400 mM, about 500 mM, about 600 mM, about 700 mM, about 800 mM, about 900 mM, about 1000 mM, about 1 M, about 1.1 M, about 1.2 M, about 1.3 M, about 1.4 M, about 1.5 M, about 1.6 M, about 1.7 M, about 1.8 M, about 1.9 M, about 2 M, about 3 M, about 4 M, about 5 M, about 6 M, about 7 M, about 8 M, about 9 M, about 10 M, about 15 M, about 20 M, about 25 M, about 30 M, about 35 M, about 40 M, about 45 M, about 50 M, about 75 M, about 100 M, or any range in between any two of the aforementioned concentrations, including said two concentrations as endpoints of the range, or any number in between any two of the aforementioned concentrations.

In some non-limiting methods, the patient is administered the AEx every one, two, three, or four weeks, for example. The dosage depends on the frequency of administration, condition of the patient, response to prior treatment (if any), whether the treatment is prophylactic or therapeutic, and whether the disorder is acute or chronic, among other factors.

Administration can be (for example but not by way of limitation) parenteral, intravenous, oral, subcutaneous, intra-arterial, intracranial, intrathecal, intraperitoneal, topical, intranasal, or intramuscular. Administration can also be localized directly into a tumor. Administration into the systemic circulation by intravenous or subcutaneous administration is typical. Intravenous administration can be, for example (but not by way of limitation), by infusion over a period such as (but not limited to) 30-90 min or by a single bolus injection.

The number of dosages administered depends on the severity of the condition and the response to therapy (e.g., whether presenting acute or chronic symptoms) Treatment can be repeated for recurrence of an acute disorder or acute exacerbation. For chronic disorders, the AEx can be administered at regular intervals, such as (but not limited to) weekly, fortnightly, monthly, quarterly, every six months for at least 1, 5, or 10 years, or for the life of the patient if the condition is chronic.

In certain non-limiting embodiments, pharmaceutical compositions for parenteral administration are sterile, substantially isotonic, and manufactured under GMP conditions. Pharmaceutical compositions can be provided in unit dosage form (i.e., the dosage for a single administration). Pharmaceutical compositions can be formulated using one or more physiologically acceptable carriers, diluents, excipients, or auxiliaries. The formulation depends on the route of administration chosen. For injection, the AEx can be formulated in aqueous solutions, such as (but not limited to) in physiologically compatible buffers such as Hank's solution, Ringer's solution, or physiological saline or acetate buffer (to reduce discomfort at the site of injection). The solution can contain formulatory agents such as suspending, stabilizing and/or dispersing agents. Alternatively, the AEx can be in lyophilized form for constitution with a suitable vehicle, e.g., sterile pyrogen-free water, before use.

Some non-limiting embodiments provided herein include kits. In some non-limiting embodiments, a kit can include a quantity of an AEx as described or otherwise contemplated herein. In some non-limiting embodiments, the AEx is lyophilized. In some non-limiting embodiments, the AEx is in aqueous solution, or other carrier as described herein. In some non-limiting embodiments, the kit includes a pharmaceutical carrier for administration of the AEx. Certain non-limiting embodiments of the present disclosure include kits containing components suitable for treatments or diagnosis. Exemplary kits may contain at least one AEx. A device capable of delivering the kit components by injection, for example, a syringe for subcutaneous injection, may be included in some non-limiting embodiments. Where transdermal administration is used, a delivery device such as hollow microneedle delivery device may be included in the kit in some non-limiting embodiments. Exemplary transdermal delivery devices are known in the art, such as (but not limited to) a hollow Microstructured Transdermal System (e.g., 3M Corp.), and any such known device may be used. The kit components may be packaged together or separated into two or more containers. In some non-limiting embodiments, the containers may be vials that contain sterile, lyophilized formulations of a composition that are suitable for reconstitution. A kit may also contain one or more buffers suitable for reconstitution and/or dilution of other reagents. Alternatively, the AEx may be delivered and stored as a liquid formulation. Other containers that may be used include, but are not limited to, a pouch, tray, box, tube, or the like. Kit components may be packaged and maintained sterilely within the containers. Another component that can be included is instructions for the use of the kit for treatment.

Examples of exosomes that can be used to create the ASL-exosomes of the present disclosure include but are not limited to those shown in Table 1.

TABLE 1
Examples of Exosomes

| Name | Company | Use | Phase |
|---|---|---|---|
| exoSTING ™ | CODIAK | Solid tumor | Phase ½ clinical trial |
| exoIL-12 ™ | CODIAK | Cutaneous T cell lymphoma | Phase 1 clinical trial |
| exoASO ™-STAT6 | CODIAK | M2 polarized tumor-associated macrophages | |
| Fortrexo | Exopharm | Antiviral | Discovery |
| Cognevo | Exopharm | Neurodegeneration | Discovery |
| PlexoDOX | Exopharm | Cancer | Preclinical |
| AB126* | ARUNABIO | Stroke | IND enabling |
| Ab127 | ARUNABIO | Gene knockdown | Proof of concept |
| Ab128 | ARUNABIO | Protein delivery | Proof of concept |
| Ab129 | ARUNABIO | Gene knockdown | Research |
| Exosome mRNA vaccine | Capricor | SARS-CoV-2 | Preclinical |
| Engineered exosomes | Capricor | Evaluating | Preclinical |
| CDC-exosomes | Capricor | Duchenne Muscular Dystrophy | Preclinical |
| ASTEX-exosomes | Capricor | Evaluating | Discovery |
| Exosome VLP | Capricor | Evaluating | Discovery |

Examples of anchor molecules that can be used to create the ASL conjugates of the present disclosure include but are not limited to Nile Blue Acrylamide, BODIPY, BODIPY™ FL NHS Ester (Succinimidyl Ester), BODIPY™ FL-05 NHS Ester (Succinimidyl Ester), BODIPY™ 493/503 NHS Ester (Succinimidyl Ester), BODIPY™ 530/550 NHS Ester (Succinimidyl Ester), BODIPY™ 558/568 NHS Ester (Succinimidyl Ester), BODIPY™ 576/589 NHS Ester (Succinimidyl Ester), BODIPY™ 581/591 NHS Ester (Succinimidyl Ester), BODIPY™ FL-X NHS Ester (Succinimidyl Ester), BODIPY™ TMR-X NHS Ester (Succinimidyl Ester), BODIPY™ TR-X NHS Ester (Succinimidyl Ester), BODIPY™ 630/650-X NHS Ester (Succinimidyl Ester), BODIPY™ 650/665-X NHS Ester (Succinimidyl Ester), Sudan 3, Nile red, Nile blue, DiL stain, DiO stain, DiR stain, DiA stain, DiB stain, and DiD stain.

Examples of spacer molecules that can be used to create the ASL conjugates of the present disclosure include but are not limited to Carboxy-PEG-Amine Compound, CA(PEG)4 Carboxy-PEG-Amine Compound, CA(PEG)8 Carboxy-PEG-Amine Compound, CA(PEG)12 Carboxy-PEG-Amine Compound, CA(PEG)24 Carboxy-PEG-Amine Compound, NH₂-PEG5K—COOH, NH₂-PEG2K—COOH, and NH₂-PEG10K—COOH.

Examples of ligand molecules that can be used to create the ASL conjugates of the present disclosure include, but are not limited to, Anisamide, Phenyl boronic acid (3-Aminophenyl boronic acid), Hyaluronic acid, Folic Acid, Cyclo(Arg-Gly-Asp-D-Phe-Lys), Transferrin, IL4RPep-1, AS-1411, GBI-10, annexins, and cetuximab.

Active agents which may be encapsulated (loaded) within the exosomes include anti-infective drugs such as quinolones (such as nalidixic acid, cinoxacin, ciprofloxacin and norfloxacin and the like), sulfonamides (e.g., sulfanilamide, sulfadiazine, sulfamethaoxazole, sulfisoxazole, sulfacetamide, and the like), aminoglycosides (e.g., streptomycin, gentamicin, tobramycin, amikacin, netilmicin, kanamycin, and the like), tetracyclines (such as chlortetracycline, oxytetracycline, methacycline, doxycycline, minocycline and the like), para-aminobenzoic acid, diaminopyrimidines (such as trimethoprim, often used in conjunction with sulfamethoxazole, pyrazinamide, and the like), penicillins (such as penicillin G, penicillin V, ampicillin, amoxicillin, bacampicillin, carbenicillin, carbenicillin indanyl, ticarcillin, azlocillin, mezlocillin, piperacillin, and the like), penicillinase resistant penicillin (such as methicillin, oxacillin, cloxacillin, dicloxacillin, nafcillinand the like), first generation cephalosporins (such as cefadroxil, cephalexin, cephradine, cephalothin, cephapirin, cefazolin, and the like), second generation cephalosporins (such as cefaclor, cefamandole, cefonicid, cefoxitin, cefotetan, cefuroxime, cefuroxime axetil, cefinetazole, cefprozil, loracarbef, ceforanide, and the like), third generation cephalosporins (such as cefepime, cefoperazone, cefotaxime, ceftizoxime, ceftriaxone, ceftazidime, cefixime, cefpodoxime, ceftibuten, and the like), other beta-lactams (such as imipenem, meropenem, aztreonam, clavulanic acid, sulbactam, tazobactam, and the like), beta-lactamase inhibitors (such as clavulanic acid), chloramphenicol, macrolides (such as erythromycin, azithromycin, clarithromycin, and the like), lincomycin, clindamycin, spectinomycin, polymyxin B, polymixins (such as polymyxin A, B, C, D, E₁ (colistin A), or E₂ (colistin B) and the like) vancomycin, bacitracin, isoniazid, rifampin, ethambutol, ethionamide, aminosalicylic acid, cycloserine, capreomycin, sulfones (such as dapsone, sulfoxone sodium, and the like), clofazimine, thalidomide, or any other antibacterial agent that can be lipid encapsulated. Anti-infectives can include antifungal agents, including polyene antifungals (such as amphotericin B, nystatin, natamycin, and the like), flucytosine, imidazoles (such as miconazole, clotrimazole, econazole, ketoconazole, and the like), triazoles (such as itraconazole, fluconazole, and the like), griseofulvin, terconazole, butoconazole ciclopirax, ciclopirox olamine, haloprogin, tolnaftate, naftifine, terbinafine, or any other antifungal that can be lipid encapsulated or complexed and pharmaceutically acceptable salts thereof and combinations thereof.

Other active agents which may be encapsulated within the exosomes include antibiotic drugs such as ampicillin, bacampicillin, carbenicillin indanyl, mezlocillin, piperacillin, ticarcillin, amoxicillin-clavulanic acid, ampicillin-sulbactam, benzylpenicillin, cloxacillin, dicloxacillin, methicillin, oxacillin, penicillin g, penicillin v, piperacillin tazobactam, ticarcillin clavulanic acid, nafcillin, cephalosporin i generation antibiotics, cefadroxil, cefazolin, cephalexin, cephalothin, cephapirin, cephradine cefaclor, cefamandol, cefonicid, cefotetan, cefoxitin, cefprozil, ceftmetazole, cefuroxime, loracarbef, cefdinir, ceftibuten, cefoperazone, cefixime, cefotaxime, cefpodoxime proxetil, ceftazidime, ceftizoxime, ceftriaxone, azithromycin, clarithromycin, clindamycin, dirithromycin, erythromycin, lincomycin, troleandomycin, cinoxacin, ciprofloxacin, enoxacin, gatifloxacin, grepafloxacin, levofloxacin, lomefloxacin, mozzxifloxacin, nalidixic acid, norfloxacin, ofloxacin, sparfloxacin, trovafloxacin, oxolinic acid, gemifloxacin, perfloxacin, imipenem-cilastatin, meropenem, aztreonam, amikacin, gentamicin, kanamycin, neomycin, netilmicin, streptomycin, tobramycin, paromomycin, teicoplanin, vancomycin, demeclocycline, doxycycline, methacycline, minocycline, oxytetracycline, tetracycline, chlortetracycline, mafenide, silver sulfadiazine, sulfacetamide, sulfadiazine, sulfamethoxazole, sulfasalazine, sulfisoxazole, trimethoprim-sulfamethoxazole, sulfamethizole, rifabutin, rifampin, rifapentine, linezolid, streptogramins, quinopristin dalfopristin, bacitracin, chloramphenicol, fosfomycin, isoniazid, methenamine, metronidazol, mupirocin, nitrofurantoin, nitrofurazone, novobiocin, polymyxin, spectinomycin, trimethoprim, colistin, cycloserine, capreomycin, ethionamide, pyrazinamide, para-aminosalicyclic acid, erythromycin ethylsuccinate, and combinations thereof

The active agent which may be encapsulated within the exosomes may be a “β-lactam antibiotic”, i.e., an antibiotic agent that has a β-lactam ring or derivatized β-lactam ring in its molecular structure. Examples of β-lactam antibiotics include but are not limited to, penams, including but not limited to, penicillin, benzathine penicillin, penicillin G, penicillin V, procaine penicillin, ampicillin, amoxicillin, Augmentin® (amoxicillin+clavulanic acid), methicillin, cloxacillin, dicloxacillin, flucloxacillin, nafcillin, oxacillin, temocillin, mecillinam, carbenicillin, ticarcillin, and azlocillin, mezlocillin, piperacillin, Zosyn® (piperacillin+tazobactam); cephems, including but not limited to, cephalosporin C, cefoxitin, cephalosporin, cephamycin, cephem, cefazolin, cephalexin, cephalothin, cefaclor, cefamandole, cefuroxime, cefotetan, cefoxitin, cefixime, cefotaxime, cefpodoxime, ceftazidime, ceftriaxone, cefepime, cefpirome, and ceftaroline; carbapenems and penems including but not limited to, biapenem, doripenem, ertapenem, earopenem, imipenem, primaxin, meropenem, panipenem, razupenem, tebipenem, and thienamycin; and monobactams including but not limited to, aztreonam, tigemonam, nocardicin A, and tabtoxinine β-lactam.

Other active agents which may be encapsulated within the exosomes include, in general, alkylating agents, anti-proliferative agents, tubulin binding agents and the like, for example, the anthracycline family of drugs, the vinca drugs, the mitomycins, the bleomycins, the cytotoxic nucleosides, the pteridine family of drugs, diynenes, and the podophyllotoxins. Examples of those groups include, adriamycin, carminomycin, daunorubicin, aminopterin, methotrexate, methopterin, dichloromethotrexate, mitomycin C, porfiromycin, 5-fluorouracil, 6-mercaptopurine, cytosine arabinoside, podophyllotoxin, or podophyllotoxin derivatives such as etoposide or etoposide phosphate, melphalan, vinblastine, vincristine, leurosidine, vindesine, leurosine and the like. The drug may be selected from camptothecin, homocamptothecin, colchicine, combretastatin, dolistatin, doxorubicin, methotrexate, podophyllotixin, rhizoxin, rhizoxin D, a taxol, paclitaxol, CC1065, or a maytansinoid, and derivatives and analogs thereof.

Other active agents which may be encapsulated within the exosomes include antineoplastic agents such as Acivicin; Aclarubicin; Acodazole Hydrochloride; Acronine; Adozelesin; Adriamycin; Aldesleukin; Altretamine; Ambomycin; A. metantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Camptothecin; Caracemide; Carbetimer; Carboplatin; Carmustine; Carubicin Hydrochloride; Carzelesin; Cedefingol; Chlorambucil; Cirolemycin; Cisplatin; Cladribine; Colchicine; Combretestatin A-4; Crisnatol Mesylate; Cyclophosphamide; Cytarabine; Dacarbazine; DACA (N-[2-(Dimethyl-amino)ethyl]acridine-4-carboxamide); Dactinomycin; Daunorubicin Hydrochloride; Daunomycin; Decitabine; Dexormaplatin; Dezaguanine; Dezaguanine Mesylate; Diaziquone; Docetaxel; Dolasatins; Doxorubicin; Doxorubicin Hydrochloride; Droloxifene; Droloxifene Citrate; Dromostanolone Propionate; Duazomycin; Edatrexate; Eflornithine Hydrochloride; Ellipticine; Elsamitrucin; Enloplatin; Enpromate; Epipropidine; Epirubicin Hydrochloride; Erbulozole; Esorubicin Hydrochloride; Estramustine;

Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; 5-FdUMP; Flurocitabine; Fosquidone; Fostriecin Sodium; Gemcitabine; Gemcitabine Hydrochloride; Gold Au 198; Homocamptothecin; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Mertansine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; PeploycinSulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Rhizoxin; Rhizoxin D; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiocolchicine; Thiamiprine; Thioguanine; Thiotepa; Thymitaq; Tiazofurin; Tirapazamine; Tomudex; TOP53; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine; Vinblastine Sulfate; Vincristine; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride; 2-Chlorodeoxyadenosine; 2′ Deoxyformycin; 9-aminocamptothecin; raltitrexed; N-propargyl-5,8-dideazafolic acid; 2-chloro-2′-arabino-fluoro-2′-deoxyadenosine; 2-chloro-2′-deoxyadenosine; anisomycin; trichostatin A; hPRL-G129R; CEP-751; linomide; sulfur mustard; nitrogen mustard (mechlorethamine); cyclophosphamide; melphalan; chlorambucil; ifosfamide; busulfan; N-methyl-N-nitrosourea (MNU); N, N′—Bis (2-chloroethyl)-N-nitrosourea (BCNU); N-(2-chloroethyl)-N′ cyclohexyl-N-nitrosourea (CCNU); N-(2-chloroethyl)-N′—(trans-4-methylcyclohexyl-N-nitrosourea (MeCCNU); N-(2-chloroethyl)-N′-(diethyl) ethylphosphonate-N-nitrosourea (fotemustine); streptozotocin; diacarbazine (DTIC); mitozolomide; temozolomide; thiotepa; mitomycin C; AZQ; adozelesin; Cisplatin; Carboplatin; Ormaplatin; Oxaliplatin; C1-973; DWA 2114R; JM216; JM335; Bis (platinum); tomudex; azacitidine; cytarabine; gemcitabine; 6- Mercaptopurine; 6-Thioguanine; Hypoxanthine; teniposide 9-amino camptothecin; Topotecan; CPT-11; Doxorubicin; Daunomycin; Epirubicin; darubicin; mitoxantrone; losoxantrone; Dactinomycin (Actinomycin D); amsacrine; pyrazoloacridine; all-trans apthal; 14-hydroxy-retro-retinol; all-trans retinoic acid; N-(4-Hydroxyphenyl) retinamide; 13-cis retinoic acid; 3-Methyl TTNEB; 9-cis retinoic acid; fludarabine (2-F-ara-AMP); or 2-chlorodeoxyadenosine (2-Cda).

Other active agents which may be encapsulated within the exosomes include, but are not limited to, 20-pi-1,25 dihydroxyvitamin D3; 5-ethynyluracil; abiraterone; aclarubicin; acylfulvene; adecypenol; adozelesin; aldesleukin; all-tyrosine kinase antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; argininedeaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; basic fibroblast growth factor (bFGF) inhibitor, bicalutamide; bisantrene; bisaziridinylspermine; bisnafide; bistratene A; bizelesin; breflate; bleomycin A2; bleomycin B2; bropirimine; budotitane; buthionine sulfoximine; calcipotriol; calphostin C; camptothecin derivatives (e. g., 10-hydroxy-camptothecin); canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; and cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; 2′ deoxycoformycin (DCF); deslorelin; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; discodermolide; docosanol; dolasetron; doxifluridine; droloxifene; dronabinol; duocarmycin SA; ebselen; ecomustine; edelfosine; edrecolomab; eflornithine; elemene; emitefur; epirubicin; epothilones; epithilones; epristeride; estramustine analogue; estrogen agonists; estrogen antagonists; etanidazole; etoposide; etoposide 4′-phosphate (etopofos); exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; homoharringtonine (HHT); hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol; irinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maytansine; mannostatin A; marimastat; masoprocol; maspin; matrilysin inhibitors; matrix metalloproteinase inhibitors; menogaril; merbarone; meterelin; methioninase; metoclopramide; MIF inhibitor; ifepristone; miltefosine; mirimostim; mismatched double stranded RNA; mithracin; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues and derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; podophyllotoxin; porfimer sodium; porfiromycin; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; rapamycin; ras farnesyl protein transferase inhibitors; ras inhibitors; ras-GAP inhibitor, retelliptine demethylated; rhenium Re 186 etidronate; rhizoxin; ribozymes; RII retinamide; rogletimide; rohitukine; romurtide; roquinimex; rubiginone B 1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene dichloride; topotecan; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

Other active agents which may be encapsulated within the exosomes include antiproliferative agents, for example piritrexim isethionate, or an antiprostatic hypertrophy agent such as, for example, sitogluside, a benign prostatic hyperplasia therapy agent such as, for example, tamsulosin hydrochloride, or a prostate growth inhibitor such as, for example, pentomone.

The term “cancer,” as used herein, may be used to describe a solid tumor, metastatic cancer, or non-metastatic cancer. In certain embodiments, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; malignant gastrinoma; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; malignant thymoma; malignant ovarian stromal tumor; malignant thecoma; malignant granulosa cell tumor; malignant androblastoma; malignant sertoli cell carcinoma; malignant leydig cell tumor; malignant lipid cell tumor; malignant paraganglioma; malignant extra-mammary paraganglioma; malignant pheochromocytoma; glomangiosarcoma; malignant melanoma; amelanotic melanoma; superficial spreading melanoma; malignant melanoma in giant pigmented nevus; epithelioid cell melanoma; malignant blue nevus; sarcoma; fibrosarcoma; malignant fibrous histiocytoma; myxosarcoma; liposarcoma; leiomyosarcoma; rhabdomyosarcoma; embryonal rhabdomyosarcoma; alveolar rhabdomyosarcoma; stromal sarcoma; malignant mixed tumor; mullerian mixed tumor; nephroblastoma; hepatoblastoma; carcinosarcoma; malignant mesenchymoma; malignant brenner tumor; malignant phyllodes tumor; synovial sarcoma; malignant mesothelioma; dysgerminoma; embryonal carcinoma; malignant teratoma; malignant struma ovarii; choriocarcinoma; malignant mesonephroma; hemangiosarcoma; malignant hemangioendothelioma; Kaposi's sarcoma; malignant hemangiopericytoma; lymphangiosarcoma; osteosarcoma; juxtacortical osteosarcoma; chondrosarcoma; malignant chondroblastoma; mesenchymal chondrosarcoma; giant cell tumor of bone; Ewing's sarcoma; malignant odontogenic tumor; ameloblastic odontosarcoma; malignant ameloblastoma; ameloblastic fibrosarcoma; pinealoma; chordoma; malignant glioma; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; malignant meningioma; neurofibrosarcoma; malignant neurilemmoma; malignant granular cell tumor; malignant lymphoma; Hodgkin's disease; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

As described above, AMD has a variety of underlying factors, including formation of lipid-rich deposits, formation of toxic byproducts, oxidation, inflammation, neovascularization and cell death. One or more therapeutic agents targeting one or more underlying factors of AMD, or having different mechanisms of action, can be utilized for the treatment of AMD. Therefore, other active agents which may be encapsulated within the exosomes include Therapeutic agents for treating AMD, such as but not limited to: anti-VEGF agents, anti-neovascularization agents, anti-dyslipidemic agents; PPARα agonists, PPARΔ agonists and PPARγ agonists; anti-amyloid agents and inhibitors of other toxic substances (e.g., aldehydes); inhibitors of lipofuscin or components thereof; visual/light cycle modulators and dark adaptation agents; antioxidants; neuroprotectors (neuroprotectants); apoptosis inhibitors and necrosis inhibitors; C-reactive protein (CRP) inhibitors; inhibitors of the complement system or components (e.g., proteins) thereof; inhibitors of inflammasomes; anti-inflammatory agents; immunosuppressants; modulators (inhibitors and activators) of matrix metalloproteinases (MMPs) and other inhibitors of cell migration; anti-angiogenic agents; laser therapies, photodynamic therapies and radiation therapies; agents that preserve or improve the health of the endothelium and/or the blood flow of the vascular system of the eye; and cell (e.g., RPE cell) replacement therapies. A particular therapeutic agent may exert more than one biological or pharmacological effect and may be classified in more than one category.

In certain embodiments, the ASL-exosomes for use in treating ocular disorders such as wet AMD could be loaded with active agents such as, but not limited to, Bevacizumab, Ranibizumab, Aflibercept, Sorafenib, Nilotinib, Abicipar, Pazopanib, Dasatinib, Sunitinib, Telatinib, OPT-302, Faricimab, Tivozanib, Ramucirumab, Vantetanib, Regorafenib, Cabozantinib, Lenvatinib, Ponatinib, Axitinib, and Brolucizumab-dbII. Among the ocular disorders that can be treated are, in non-limiting examples, wet age-related macular degeneration (wet AMD), macular edema, diabetic retinopathy, and retinal vein inclusion.

Returning now to the description of particular non-limiting embodiments of the present disclosure, the ASL system synergistically integrates the following functions. First, the physical anchoring using a hydrophobic/lipophilic dye allows exosomes to overcome current challenges associated with surface modification methods such as chemical conjugations and transfection of exosome-producing cells. Second, the present anchoring method is not only fast and easy to control but also safely preserves exosome structure and function while offering an additional trackable diagnostic modality. Third, the ASL-based targeting moieties will enable exosomes to preferentially target disease sites thus minimizing systemic off-target issues, particularly associated with current standard cancer therapies. Together, the modularity of the ASL for modifying exosomes elevates AExs into a platform technology with widespread applicability, where AExs can be engineered with hydrophobic/lipophilic anchors of desired fluorescence characteristics coupled with a targeting ligand of choice for specific delivery to disease sites and cells, including non-cancerous disease sites and cells.

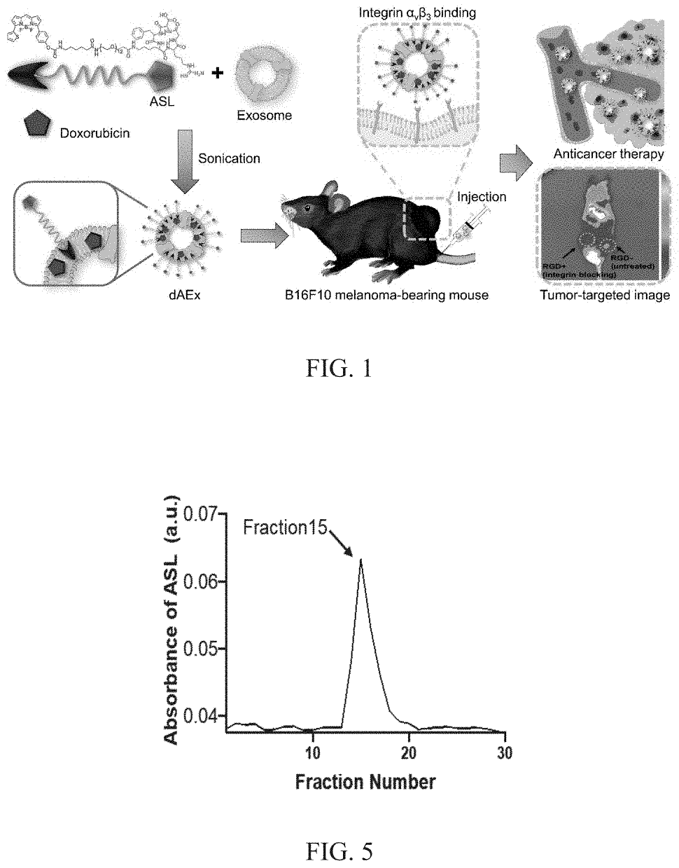

In one embodiment, as described in Example 1, active targeting exosomes (AEx) were prepared by synthesizing ASL conjugates with a BODIPY anchor, a PEG spacer, and an RGD targeting ligand (FIG. 1). AEx were loaded with the drug doxorubicin (DOX) to form dAEx. Once the physicochemical properties of the ASL conjugates, AEx, and dAEx, were characterized, the impact of ASL on targeted delivery and therapeutic efficacy of dAExs were validated using melanoma cells (B16F10) in in vitro and in vivo studies. ASL system is a one-of-a-kind platform for utilizing cell membrane staining dyes as an anchor conjugated to a targeting ligand separated by a PEG spacer. This innovative platform provides a new class of engineered exosomes, AExs, for clinical therapy. In a particular non-limiting embodiment of the present disclosure, the specific interaction between RGD and αᵥβ₃ integrin will allow specific accumulation of a drug (e.g., DOX) encapsulating ASL exosomes (e.g., dAEx) and delivery of the chemotherapeutic agent to the tumor microenvironment.

In another embodiment, as described in Example 2, AEx were prepared by synthesizing ASL conjugates with a BODIPY anchor, a PEG spacer, and an RGD targeting ligand then were loaded with the drug Aflibercept. The loaded AEx were effectively delivered to the area of retinal diseases with active targeting, and effectively suppressed ocular neovascularization more effectively than direct intravitreal injection of drug alone.

Having generally described embodiments drawn to targeted exosomes which comprise ASL conjugates, a further understanding of the compositions and methods of the present disclosure can be obtained by reference to certain specific examples which are provided below for purposes of illustration only and are not intended to be limiting.

### Example 1

### In Vivo Tumor-Targeting of Exosomes

### Chemicals and Reagents

BODIPY TR-X NHS Ester and Carboxy-poly(ethylene glycol) (PEG)12-Amine compound were purchased from Thermo Fisher Scientific (Rockford, Ill., USA). Cyclic (Arg-Gly-Asp-D-Phe-Lys) (RGD) was obtained from Peptide International (Louisville, Ky., USA). Dichloromethane (DCM), dimethyl sulfoxide (DMSO), diethyl ether, and hexanes were obtained from VWR International (Radnor, Pa., USA). Doxorubicin hydrochloride, triethylamine (TEA), 1-Ethyl-3-(3-dimethylaminopropyl) carbodiimide (EDC), N-Hydroxysuccinimide (NETS) were purchased from Sigma-Aldrich (St. Louis, Mo., USA).

### Synthesis of ASLs

BODIPY TR-X NHS Ester (5 mg) and TEA (1.53 μL) were dissolved in DCM (1 mL) at room temperature. Carboxy-PEG12-Amine (4.87 mg) was added into the mixture. The mixture was stirred vigorously for six hours for reaction, and compound 1 was obtained as a purple powder from precipitation in cold diethyl ether. The chemical structure of compound 1 was confirmed by ¹H NMR in D20 on an automated Bruker 400 MHz spectrometer. The compound 1 (10 mg), EDC (7.97 mg), and NETS (1.2 mg) were completely dissolved in 1 mL of DMSO followed by addition of TEA (0.17 μL), and the mixture was stirred for 1 hour. Cyclic RGD (7.87 mg) was dissolved in 0.3 mL of DMSO and added to the mixture. The reaction was allowed to continue overnight at room temperature. The residual cyclic RGD, EDC, NETS, and DMSO were removed by membrane dialysis tubing (Cutoff 1\4W 1000, Spectrum Laboratories, Inc., Rancho Dominguez, Calif., USA) and purified by SEC using a SEPHACRYL® S-100 HR column (Sigma-Aldrich, St. Louis, Mo., USA). ASL was obtained by lyophilization. The molecular structure of ASL was characterized by ¹H NMR, and the molecular weight was measured using MALDI-TOF analysis (Microflex, Bruker, Billerica, Mass.).

### Preparation of Hydrophobic DOX

Doxorubicin (DOX) hydrochloride (40 mg) was completely dissolved in DMSO (1 mL) under vigorous stirring, and TEA (30.77 μL) was added. The desalting process was allowed under dark conditions at room temperature. After 12 hours, the mixture solution was added into cold water to precipitate, and desalted doxorubicin was obtained by lyophilization.

Preparation and Characterization of dAEx

B16F10 cells were purchased from ATCC (Manassas, Va., USA). Cells were cultured in Dulbecco's Modified Eagle Media (DMEM) containing 10% fetal bovine serum (FBS) and incubated with 5% CO₂ at 37° C. In order to obtain exosomes, cells were cultured in serum-free DMEM for 48 h at 37° C. in 5% CO₂. Cell culture media were collected and centrifuged at 2000×g. To concentrate the supernatant, the ultrafiltration process was performed using AMICON® Ultra Centrifugal Filters (Cutoff MW 50000, Millipore Sigma, Bedford, Mass., USA). Then, the concentrated supernatant was filtered through a 0.2 μm membrane (Nalgene syringe filter, Thermo Fisher Scientific, Rockford, Ill., USA). For the preparation of DOX-AEx (dAEx), ASL (50 μg) and 100 μg of DOX in DMSO (10 μL) were added to the solution of exosomes. dEx was prepared by the addition of 100 μg of DOX in DMSO (10 μL) to the solution of exosomes. The procedure for functionalization and encapsulation was proceeded using a sonication method, and the samples were given an incubation time at 37° C. for 1 hour to recover the integrity of the exosomes membrane. dAEx and dEx were purified by an SEC. The absorption and emission spectra were recorded using a microplate reader (SpectraMax, Molecular Devices, San Jose, Calif.). The measurement of the hydrodynamic size of dAEx was carried out using a Zetasizer Nano (Malvern Panalytical, Malvern, UK), and the particle size distributions were provided by the nanoparticle tracking analysis (NanoSight LM10, Malvern Panalytical, Malvern, UK). The nanoparticles were stained using a uranyl acetate, and the shape was observed by a transmission electron microscopy (TEM, JEOL 1200EX, JEOL, Japan).

Purification of dAEx

dAEx was loaded onto the 15 cm of the SEPHACRYL® S-100 HR column and eluted using a fresh PBS. The fractions were collected every 0.5 mL per fraction and a total of 30 fractions. The absorption of DOX (488 nm) and ASL (588 nm) was measured using a microplate reader, and the elution profile of exosomes was observed using a Bradford protein assay (Bio-Rad, Hercules, Calif., USA).

Release Kinetics of dAEx

The release kinetics of DOX and ASL from dAEx or BODIPY from BODIPY-labeled Ex was conducted using the membrane dialysis tube (Cutoff MW 3500) and PBS containing 0.05% (w/v) Tween 80 as a release medium. At the given time to collect, the media was collected and replaced. The fluorescence intensity of samples was measured using a microplate reader to determine the concentrations of ASL, DOX, and BODIPY.

Integrin αᵥβ₃-binding Assay for Expression of RGD on Exosomes

Integrins (R&D systems, Minneapolis, Minn., USA) were coated overnight onto 96 wells (5 μg/mL, 50 μL/well). After incubation, wells were washed twice using the binding buffer (50 mM Tris, pH 8, 150 mM NaCl, 1 mM MgCl₂, 1 mM CaCl₂) and 0.5 mM MnCl₂). AEx and BODIPY-labeled Ex were resuspended in binding buffer and loaded to the wells. To determine the RGD-specific binding of ASL system, AEx was added to the integrin αᵥβ₃-coated wells that were treated with synthetic RGD peptide (2 μM) for 2 h. After treatment 2 h, samples were discarded and washing process was performed twice. The fluorescence signals of AEx or BODIPY were measured using a microplate reader.

### Cell Culture Studies.

Cells (B16F10 and RAW 264.7 cell line) were grown in DMEM containing 10% FBS at 37° C. with 5% CO₂. B16F10 and RAW 264.7 cells were seeded on the 96 well tissue culture plates (1×10⁴ per well) and cultured for 24 hours. To confirm the biosafety of ASL and AEx, the cells were treated with various concentrations of ASL and AEx and incubated for 24 hours. For the cytotoxicity test, B 16F10 cells were given the various concentrations of dAEx, dEx, DOX. In addition to the further competition test, B16F10 cells were incubated with the cyclic RGD (2 μM) for 2 hours and treated with the various concentrations of dAEx. After 24 hours of the treatment, to investigate the cell survival, cells were washed using the fresh PBS twice, and 10% CCK-8 containing DMEM was added to each well and incubated for 1 hour. The absorbance was measured using a microplate reader at 450 nm.

### Scanning Confocal Microscopy

B16F10 cells were seeded in a confocal dish (VWR International, Radnor, Pa., USA) at a density of 0.3×10⁶/dish and incubated for 24 hours. To block integrin αᵥβ₃, one of the dishes was incubated with the synthetic RGD peptides (2 μM) for 2 hours. Cells were treated with dEx, dAEx for 3 hours, and washed with fresh PBS three times. 4% paraformaldehyde in PBS was used as a fixing reagent, and the DAPI stock solution (1:5000 in PBS+) was treated to label the nucleus of the cells. The images were acquired using a confocal microscope (SP8 LIGHTNING Confocal Microscope, Leica, Wetzlar, Germany).

Apoptotic Activity of dAEx in Cancer Cells

B16F10 cells were seeded in a 100 mm cell culture dish with cell density at 4.4×10⁶ and incubated for 24 hours. After the treatment of various agents with a fixed concentration of DOX (5 μM) for 24 hours, proteins were collected using a cell lysis buffer on ice. Proteins were separated by electrophoresis and transferred to PVDF membranes. Cleaved caspase-3, cleaved PARP, and β-actin (Cell Signaling Technology, Danvers, Mass., USA) were used as primary antibodies, and IRDye secondary antibodies (LI-COR Biosciences, Lincoln, Nebr., USA) were used as secondary antibodies. The protein bands were imaged using the Odyssey CLx (LI-COR Biosciences, Lincoln, Nebr., USA). The apoptotic activities of dAEx were evaluated by measuring the level of cleaved caspase-3, and cleaved PARP in the protein samples with a cleaved PARP (Cell Signaling Technology, Danvers, Mass., USA) and a cleaved caspase-3 (R&D systems, Minneapolis, Minn., USA) ELISA kit according to manufacturer's protocol.

### B16F10 Murine Tumor Model

B16F10 cells (1×10⁶) were directly injected into the flank of 6 weeks old mice. The mice were randomly grouped by six in each and observed until the cancer volume reached ˜50 mm³. When tumors had developed, the mice were grouped randomly and treated through intravenous route with ASL (2.5 mg/kg), DOX (5 mg/kg), exosomes, dEx and dAEx (1.27±0.24×10¹¹/kg) at DOX dosage of 5 mg/kg every 3 days for 4 injections, and the tumor volume and body weight were observed for 13 days. The tumor volume was calculated based on the formula: (width²×length)/2. On day 18, the mice were sacrificed, and organs were harvested for histological analysis. H&E and TUNEL staining were performed using the paraffin-embedded tissues.

### In Vivo Targeting Validation

The tumor-bearing mice were fluorescently imaged at an excitation wavelength of 588 nm and an emission wavelength of 620 nm along with a high-resolution digital x-ray to deliver precise anatomical localization of molecular and cellular biomarkers in vivo. Quantitative fluorescence measurement was conducted with a multispectral imaging platform (Bruker multispectral MS FX PRO imaging system, Carestream Health, Inc.), and fluorescence intensity in the area of interest was analyzed using a Molecular Imaging software v7.5.

In Vivo Biodistribution of dAEx Using Tumor-bearing Mice

For the biodistribution study, mice were injected with dAEx or BODIPY-labeled Ex or dEx. After 3, 6, 12, or 24 hours, the tumors, organs, and blood were collected at a given time. The samples were lyophilized and resuspended in DMSO (200 mg/mL) to extract DOX and ASL. To collect supernatant, centrifugation (10,000×g for 20 min) was performed, and the supernatants were filtered. The fluorescence signals of samples were measured using a microplate reader.

### Statistical Analysis

Values were expressed as mean±standard deviation (SD). To analyze the differences among groups, one-way analysis of variance (ANOVA) was performed using GraphPad Prism 8.0.1 (San Diego, Calif.). Probability (p) values of <0.05 were considered statistically significant.

### Results

Preparation and Characterization of DOX Encapsulated ASL Exosome (dAEx)

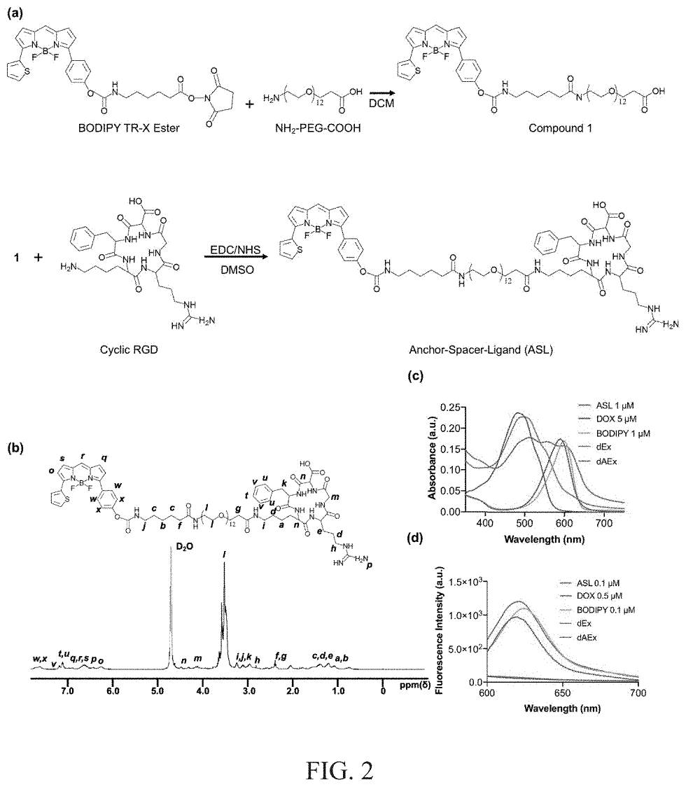

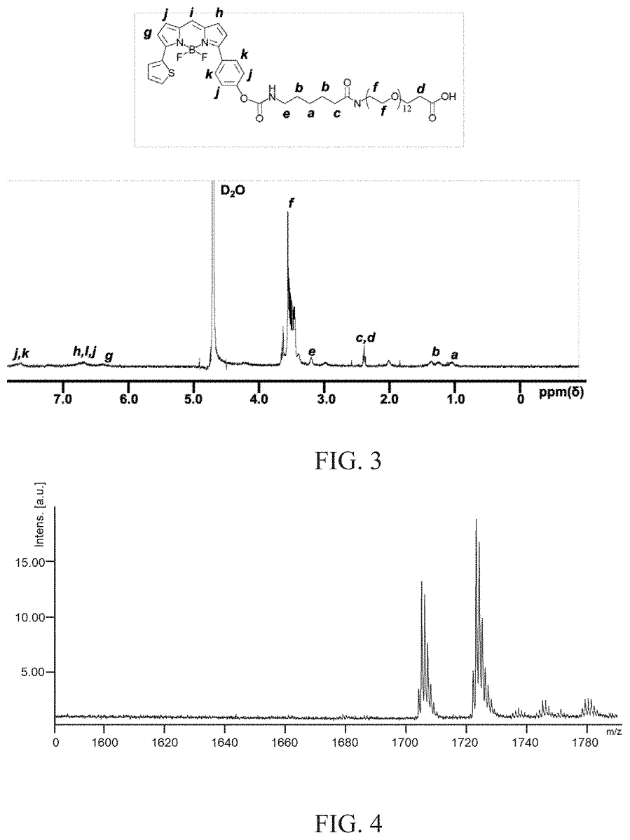

To establish dAEx, ASL was designed to endow theranostic capacities by functionalizing exosomes based on the membrane binding affinity of BODIPY. As illustrated in FIG. 2a, integrin αᵥβ₃-targetable RGD and PEG was employed to obtain ASL by conjugating BODIPY. Compound 1 was synthesized from BODIPY TR-X NHS Ester and COOH-PEG-NH₂ by a reaction of NHS esters with the amine, and chemical structure was confirmed by ¹H NMR (FIG. 3). Cyclic (Arg-Gly-Asp-D-Phe-Lys) (cyclic RGD) was coupled with compound 1 to give a stable amide bond of ASL by EDC/NHS chemistry. The chemical structure of ASL was confirmed by ¹H NMR spectroscopy (FIG. 2b), and the molecular weight of ASL was determined to be ˜1730 Da by matrix-assisted laser desorption/ionization-time of flight (MALDI-TOF) mass spectroscopy (FIG. 4). ASL after chemical conjugation were purified by size exclusion chromatography (SEC), and it was performed to assess the elution profile of ASL. SEC purification results show the highest peak that appeared in fraction 15 (FIG. 5).

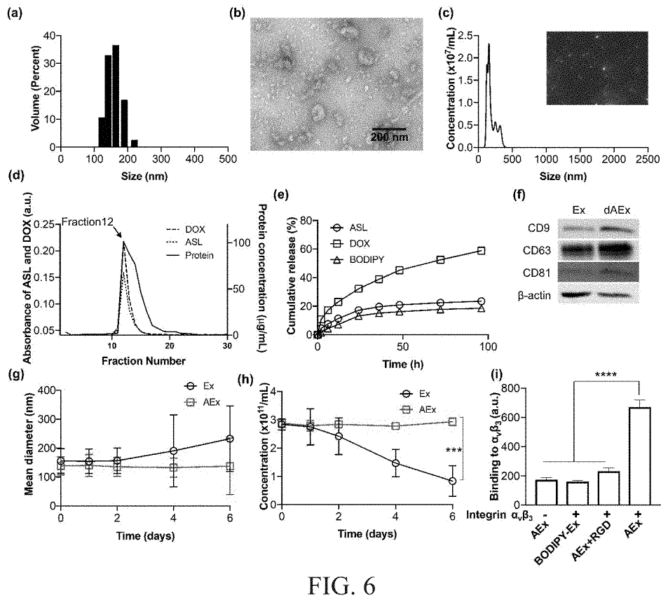

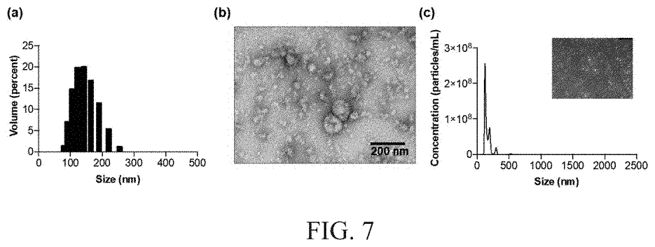

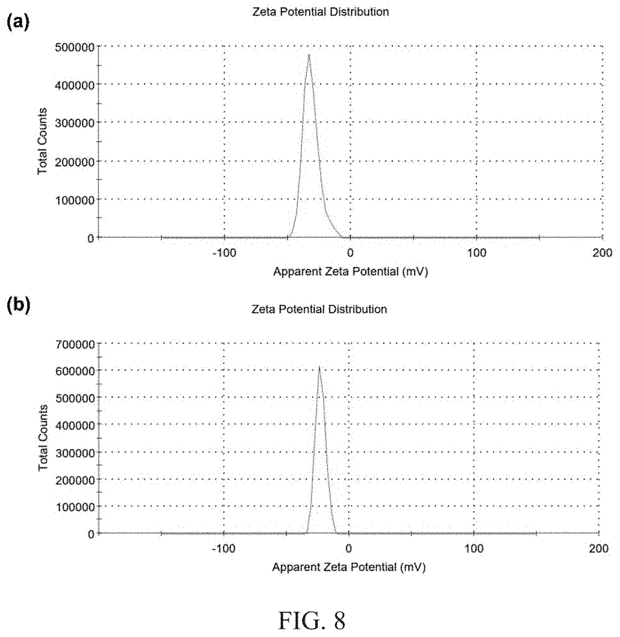

The optical properties of dAEx were investigated using absorption and fluorescence spectroscopy since DOX and ASL are incorporated within the exosome membrane. DOX showed an absorption spectrum at 488 nm, and BODPIY exhibited at 588 nm with its emission at 622 nm. After mixing ASL and DOX with exosomes, SEC purification removed unincorporated DOX and/or ASL from mixtures. SEC-purified dAEx showed the absorbance spectra of DOX and BODIPY simultaneously (FIG. 2c). Since ASL showed fluorescence at wavelengths (622 nm) similar to that of BODIPY, dAEx were also observed at 622 nm due to incorporated ASL (FIG. 2d). The incorporated amount of ASL and DOX within dAEx was determined after SEC purification. We obtained concentrations of DOX and ASL incorporated within dAExs from the calibration curve of free DOX (488 nm excitation and 565 nm emission) and free BODIPY (588 nm excitation and 630 nm emission) normalized by exosome amount and found that dAExs (˜10⁹ exosomes) incorporate 21.0±3.9 μg of ASL and 39.2±7.2 μg of DOX. The morphology of dAEx by TEM measurement shows a cup shape dAEx (FIG. 6b) which is similar to exosome (FIG. 7b) and indicating a hydrodynamic diameter of ˜171.8±24.6 nm by dynamic light scattering (DLS) and ˜170.2±10.5 nm by nanoparticle tracking analysis (NTA) (FIG. 6a,c), which is slightly larger than unmodified exosomes (DLS: 143.4±37.8 nm and NTA: ˜154.7±2.4 nm) due to ASL incorporation of dAEx (FIG. 7a,c). Zeta potential of dAEx was measured as −22.9±3.98 mV and the value changed compared to exosomes (−31.6±6.48) (FIG. 8). To detect the incorporation of ASL and DOX within dAEx, we eluted the mixture of ASL, DOX, and dAEx through the SEC column, and the fluorescence intensity of each fraction was determined. The elution profiles of ASL and DOX were found at the same fractions of dAExs, and the highest peak fractions were detected earlier than free ASL fractions (FIG. 6d), indicating that dAExs indeed incorporate ASL and DOX, and residual ASL and DOX can be separated from dAEx post-formulation through SEC purification. We also determined the release kinetics of ASL and DOX from dAEx compared with BODIPY-labeled exosomes to confirm the membrane-labeling mechanism of ASL. As shown in FIG. 6e, ASL shows near-identical release kinetics as BODIPY from exosomes, with their cumulative released amounts totaling to less than 20% even after extended incubation (˜96 hours), indicating the stability of ASL anchoring on dAEx. However, the release kinetics of DOX from dAEx was comparatively enhanced, suggesting that the membrane retention and stability of ASL is dependent on the interaction of BODIPY anchor with exosome membrane. To test whether ASL and DOX incorporation into dAExs could change the exosomal membrane integrity, expression of exosome marker such as tetraspanins (CD9, CD63, and CD81), within exosome membranes was characterized by western blot. The membrane proteins were detected both in exosomes and dAEx (FIG. 6f), demonstrating that the dAEx formulation process does not affect the exosomal membrane integrity.

Additionally, we tested the effect of ASL independently on AEx stability, especially at higher concentrations. Surprisingly, exosome particles increase in size, while AEx maintain their particle size over the course of 6 days (FIG. 6g). This phenomenon of size evolution, particularly in exosomes, is due to exosome instability and progressive fusion as noted by corresponding decrease in particle concentration (FIG. 6h). Thus, we show that ASL play a vital role in protecting exosomes against degradative exosome fusion during extended incubation, which will prove beneficial for production and storage, and, importantly, for long-term stability during circulation in vivo.

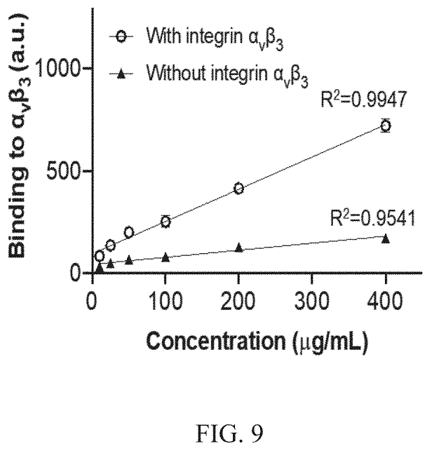

To investigate the cancer cell affinity of ASL, the binding ability of ASL to integrin αᵥβ₃ was analyzed by measuring the fluorescence intensity. On integrin-immobilized wells, ASL showed strong fluorescence intensity compared to integrin-free wells depending on the concentration of ASL (FIG. 9). AEx, ASL-engineered exosome, showed ˜4 folds stronger fluorescence signals than other groups on integrin-coated wells, but RGD treatment inhibited the binding on integrins (FIG. 6i). In these results, AEx has a strong affinity to cancer cells and the presence of RGD on the surface of AEx was confirmed. As an engineering tool for exosomes, ASL system have a great advantage as an assessment method to prove RGD expression on exosomes comparing conventional methods.

### Enhanced Anticancer Activities of ASL-modified Exosomes In Vitro

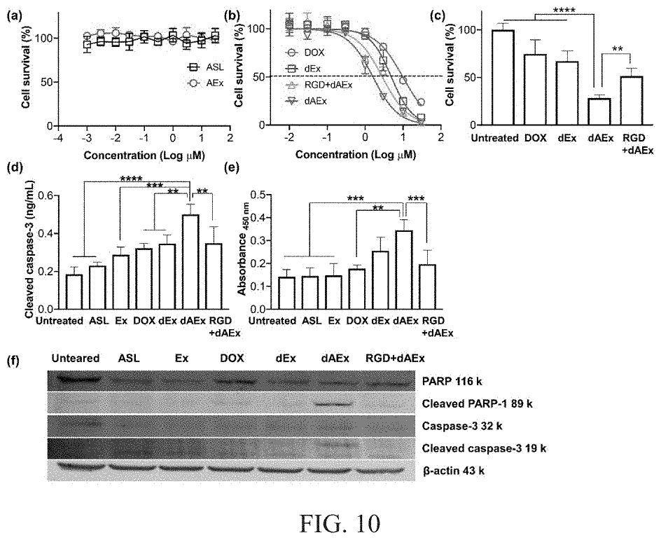

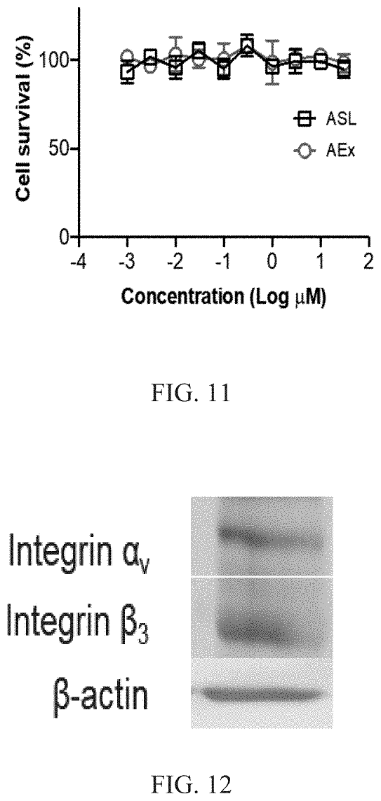

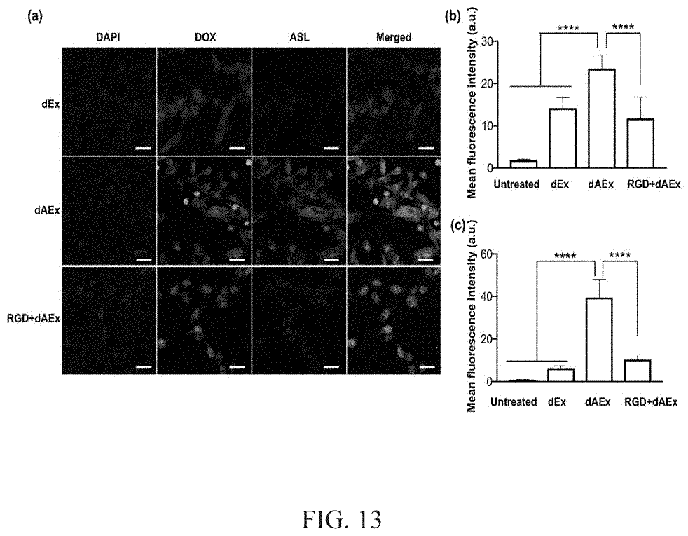

To determine optimal therapeutic administration, varying concentrations of ASL and AEx were tested for cytotoxic activity against murine macrophage (RAW 264.7) and B16F10 cells. At highest concentration (30 μM), both ASL and AEx with equivalent concentrations of ASL showed no toxicity in RAW 264.7 cells and B16F10 cells (FIG. 10a and FIG. 11, respectively). We then determined the efficacy of active targeting by dAEx by measuring the integrin αᵥβ₃-dependent cellular uptake of dAEx. First, we evaluated the expression of the RGD-binding receptors, αᵥβ₃ integrin, on B16F10 melanoma cells (FIG. 12). Then, we treated B16F10 with DOX-loaded exosomes (dEx), dAEx, or dAEx and free RGD, where addition of free RGD is anticipated to bind to αᵥβ₃ integrin competitively with dAEx (FIG. 13a). Strong fluorescence signals for ASL and DOX were observed in the dAEx-treated cells, but their fluorescence intensity was significantly inhibited by addition of exogenous RGD (2 μM) (FIG. 13b,c). These results indicate that dAEx achieves targeted binding mediated by specific interaction between its RGD ligands on ASL and integrin αᵥβ₃ on B16F10 cells. Notably, fluorescent images of cytosolic localization of DOX and ASL signals indicate that binding through integrin αᵥβ₃ leads to enhanced cellular uptake of dAEx by B16F10 cells. To evaluate the functional implications of various synthesized exosomal formulations, surface modifications, and consequently enhanced uptake, we compared the anticancer activity of dAEx against DOX, dEx, and dAEx with RGD treatment on B16F10 melanoma cells at various treatment concentrations. Similarly, with fluorescent imaging, co-treatment of free RGD (2 μM) and dAEx was studied as an experimental group to illustrate the impact of ASL targeting. dAEx exerted significantly enhanced anticancer activity, exhibiting lowest half-maximal inhibitory concentrations (IC50) at 1.606±0.255 μM, compared to those of other treatment groups (9.852±1.058 μm for DOX, 4.908±0.739 μm for dEx as a non-targeted exosome, and 2.837±0.576 μm for dAEx with free RGD) (FIG. 10b). Specifically, at the treatment dose of 3 μM concentration, dAEx showed the lowest cell survival (<30%) compared to other groups (>50%), a result attributable to ASL-mediated active targeting and corresponding enhancement in uptake and delivery of DOX to target cells (FIG. 10c).

Induction of apoptosis by dAEx was explored by comparing expressions of apoptosis-associated caspase-3 and poly (ADP ribose) polymerase (PARP) cleavage. Expression of apoptosis-associated proteins was assayed by enzyme-linked immunosorbent assay (ELISA) and western blot (FIG. 10d-f). Both ASL and exosome (Ex) showed negligible expression of cleaved caspase-3 and PARP. DOX and dEx exerted slight activation of apoptotic proteins as expected since DOX has been shown to induce programmed cell death. However, dAEx induced significant increase in cleavage of caspase-3 and PARP which was inhibited by addition of free RGD, once again demonstrating ASL-dependence by dAEx in targeted binding to and apoptosis induction on B16F10. These results are in agreement with our results from cell counting kit-8 (CCK-8) assay and confocal analysis (FIG. 10b and FIG. 13a), confirming that dAEx exhibits specific ASL-dependent active targeting of B16F10 cells, leading to enhanced uptake and, consequently, deploying cytotoxic efficacy in inhibit tumor growth.

In Vivo Validation of ASL-Mediated Enhanced Therapeutic Efficacy by dAEx

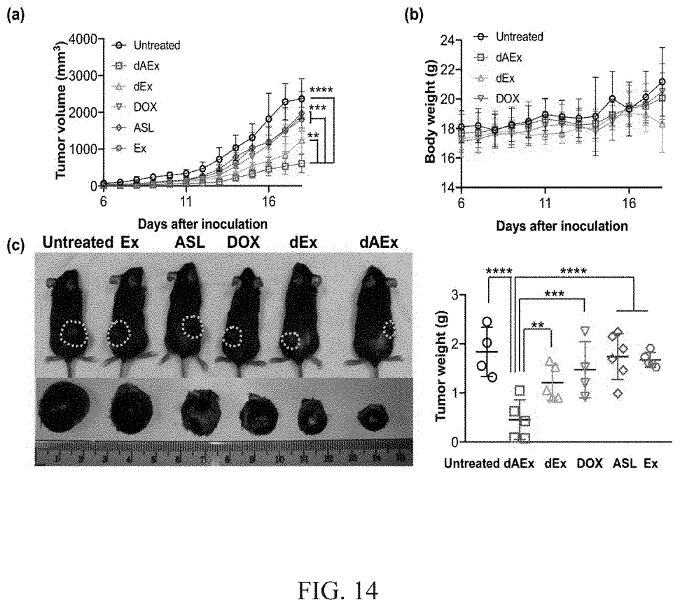

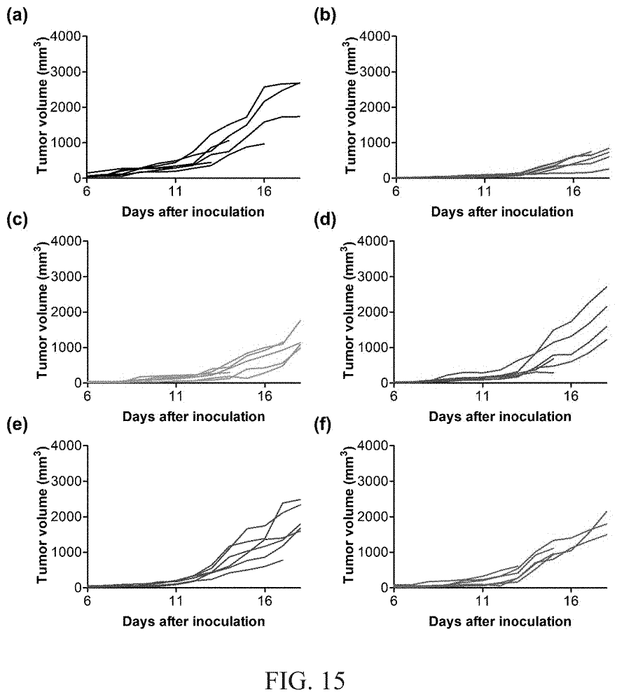

We investigated the in vivo therapeutic efficacy of dAEx using a B16F10 syngeneic mouse tumor model. When the tumor volume reached ˜50 mm³, mice were treated with ASL, Ex, DOX, dEx, or dAEx at DOX dosage of 5 mg/kg through an intravenous (IV) tail vein injection every three days. Tumor volume and body weight were observed for 18 days post-tumor inoculation. Treatment with ASL, DOX, or Ex did not significantly inhibit tumor growth compared to untreated control group. Although dEx moderately decreased tumor growth, dAEx exhibited significant suppression of tumor development exceeding efficacy shown by dEx and all other treatment groups (FIG. 14a,c). FIG. 15 shows the variation in individual tumor growth within the treatment groups (a) Untreated, (b) dAEx, (c) dEx, (d) DOX, (e), ASL, and (f) Ex, and clearly shows suppression of tumor growth in the dAEx treatment group. Also, no significant body weight change between groups shows the minimal toxicity of each treatment formulation (FIG. 14b). Tumor weight results are also consistent with tumor volume measurement, further supporting therapeutic efficacy of dAEx.

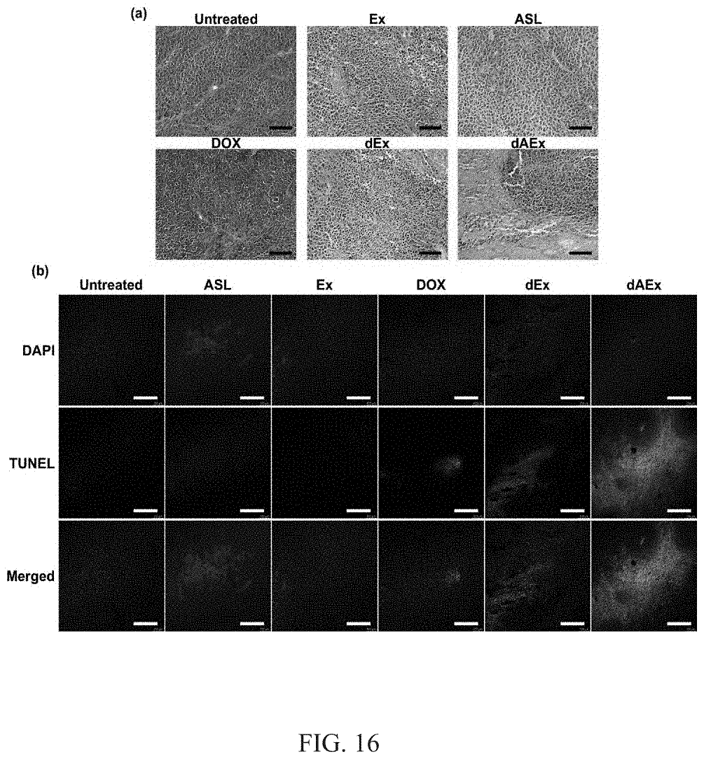

The anti-tumor efficacy of dAEx were further studied by hematoxylin and eosin (H&E) staining and terminal deoxynucleotidyl transferase dUTP nick end labeling (TUNEL) staining of excised tumors. The untreated animal's tumor tissue stained with H&E shows dense configuration, clear nucleus, and intact membrane structure (FIG. 16a). Similarly, no dead cells are observed ASL or Ex treatment groups. dEx-treated tumor tissue exhibits morphological changes in tumor cell membranes as well as cells without nucleus, drawing contrast with DOX-treated group. Notably, tumor tissue from dAEx treated animal clearly presents cellular damage, irregular composition and unhealthy nucleus in tumor cells. Tumor tissue from dAEx-treated mice also manifested with apoptotic cell death, which was evidenced by the stronger expression of TUNEL-positive signals than other treatment groups (FIG. 16b). These observations, together, indicate that dAEx enhances the therapeutic activity of DOX through targeted activity and improved drug delivery.

In Vivo Active Targeting Efficacy of dAEx

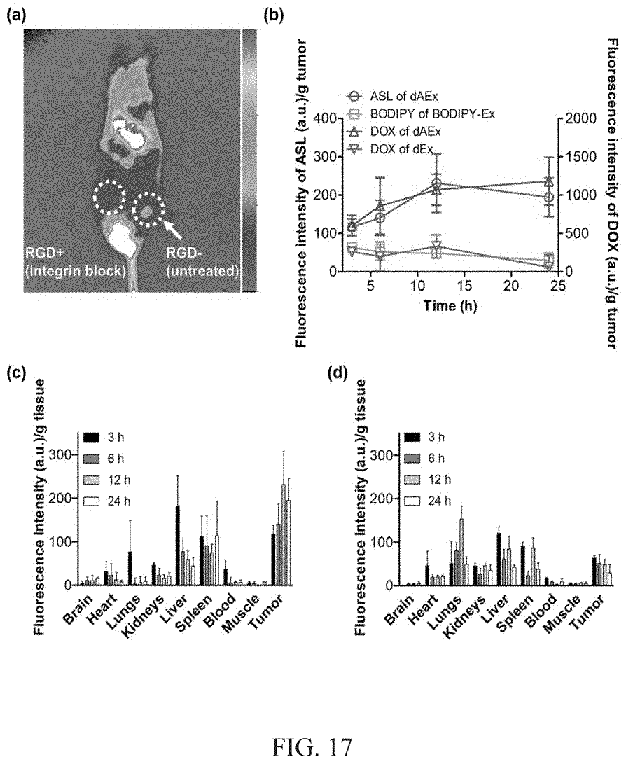

To confirm the efficacy of active targeting exosomes (dAEx) as attributable to their in vivo active tumor targeting, we inoculated B16F10 cells at both right and left flanks of mice. Using these mice, we administered dAEx through IV tail vein, followed by pre-treatment of free RGD into one side of the tumor to induce the competitive interaction between free RGD and dAEx (FIG. 17a). Then, the animals were scanned by an in vivo fluorescent imaging instrument. The images demonstrate that accumulation of dAEx at tumors pre-treated with free RGD is minimal, while the other flank-side tumor untreated with RGD showed dAEx accumulation. The competition between free RGD and dAEx-bound ASL limits the specific targeting of dAEx to B 16F10 tumor, showcasing that dAExs target tumor sites by RGD-mediated active targeting efficacy of ASL. Thus, the ASL-mediated active-targeting system disclosed herein enables exosomes to home and localize to desired disease sites. These results conclusively establish the active targeting efficacy of dAEx, consistent with the in vitro and in vivo therapeutic results demonstrating ASL-based functional modification of exosomes.

In Vivo Systemic Fate of dAEx

To study the biodistribution and possible off-target effects of dAEx and other exosome formulations upon in vivo administration, we interrogated their systemic fate in melanoma tumor-bearing mice. Upon tumor inoculation and growth to ˜50 mm³, we treated mice with BODIPY-labeled Ex, dEx, or dAEx through IV tail vein, and studied their organ-level biodistribution at each timepoint. FIG. 17b shows the time-dependent accumulation of dAEx in tumor compared with non-targeted exosomes (BODIPY-Ex or dEx). The accumulation trend of ASL in tumor was gradually increased over time, but exosomes without ligands showed low fluorescence signals, which indicate the in vivo stability and targeting of dAEx. It is supported by the similar trend of DOX accumulation results. We next determined anchor fluorescent intensity of dAExs from each organ and blood (FIG. 17c,d). Other than the tumor, dAExs also showed accumulation in the liver and spleen. Importantly, dAEx shows the highest tumor accumulation in comparison to nontargeted exosomes (BODIPY-labeled Ex), also demonstrating the advantages of active targeting enabled by ASL.

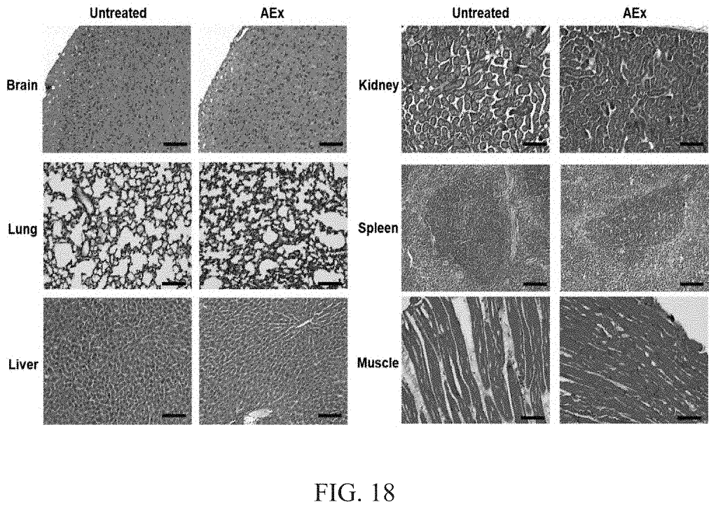

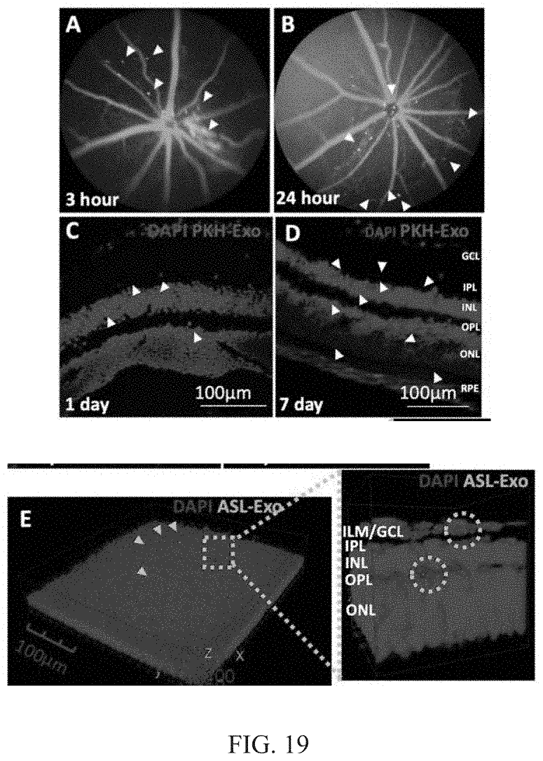

We also investigated the in vivo systemic toxicity of AEx as a drug carrier. AEx were intravenously administrated to mice once a day, and organs were collected seven days post-injection. Histological examination of each organ revealed no significant difference between the AEx and untreated group, indicating that the AEx platform presents minimal systemic toxicity (FIG. 18).

Thus, these results establish that ASL-modification allows exosomes to be engineered as stable, trackable, and target-specific delivery platforms. With their demonstrable safety profiles as demonstrated by absence of observable systemic effects, ASL-modification of exosomes as a novel trackable, targeted drug delivery platform is uniquely positioned for widespread clinical translation.

In summary, an ASL system as disclosed herein has been developed as a new tool for exosome engineering to enhance therapeutic capability of exosomes against disease conditions such as cancer. In non-limiting, proof-of-concept experiments, ASL was synthesized from BODIPY, PEG, and RGD. ASL not only gave stability but also provided cancer targetability and imaging capabilities enabling exosomes to be used as theranostic agents. ASL-engineered exosomes enhanced the therapeutic efficacies of DOX. In a mouse model of cancer, dAEx achieved tumor-targeted imaging, and significantly suppressed the tumor development without distinct side effects. ASLs can thus be utilized with exosomes for both therapeutic and diagnostic applications.

### Example 2

Intraocular Drug Delivery with Exosomes

Although active targeting strategies have been studied and employed in cancer research, active targeting strategies in ocular drug delivery have not been clinically applied. The present disclosure provides an innovative approach to shift the intraocular drug delivery platform from passive targeting-directed drug delivery to active targeting-directed drug delivery.

To develop intraocular sustained multi-drug delivery, many studies have focused on synthetic nanoparticles. However, these have not been clinically applied due to various challenges including penetration of ocular barriers, aggregation of particles in the vitreous cavity causing visual disturbance, and immune reactions to synthetic materials in the eye. The present disclosure provides modified exosomes as an intraocular drug delivery system which is superior to synthetic nanoparticles based on the characteristics of exosomes that combine nanoparticle size with a non-cytotoxic effect, target specificity, and a multi-cargo carrying capacity including MicroRNA (miR), proteins or lipids, for intercellular communications. The present results show that intravitreally-delivered exosomes can distribute to the retina in both inner and outer retina layers and also into the intracellular space. Therefore, exosome based intraocular drug delivery can benefit multi-drug delivery targeting both extracellular and intracellular targets including target gene delivery (FIG. 19).

Since exosomes are released by a variety of cell types, a number of options can be considered for the selection of a donor cell from which to isolate exosomes. Two important factors that play a role in the selection are biological properties of the exosomes and yield of exosomes from the specific cell type. We tested exosomes from two sources, primary cultured mouse Müller glia and whole retina from wild type mouse. Because there was no observable difference in in vivo retinal uptake between two types of exosomes and because whole retina-derived exosomes had a better yield and potential multi-cellular preferences within the retina, we used whole retina from wild type mouse-derived exosomes for ASL-exosome engineering.

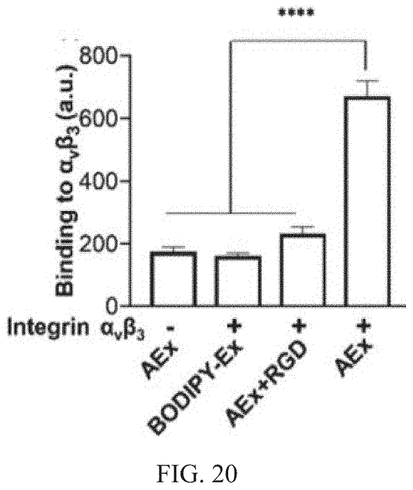

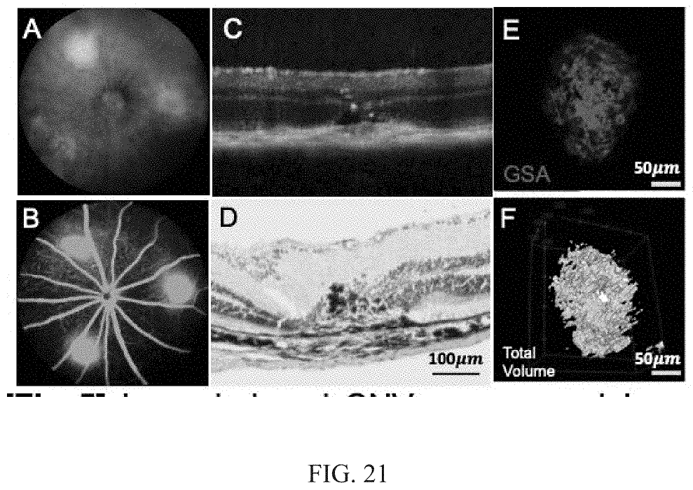

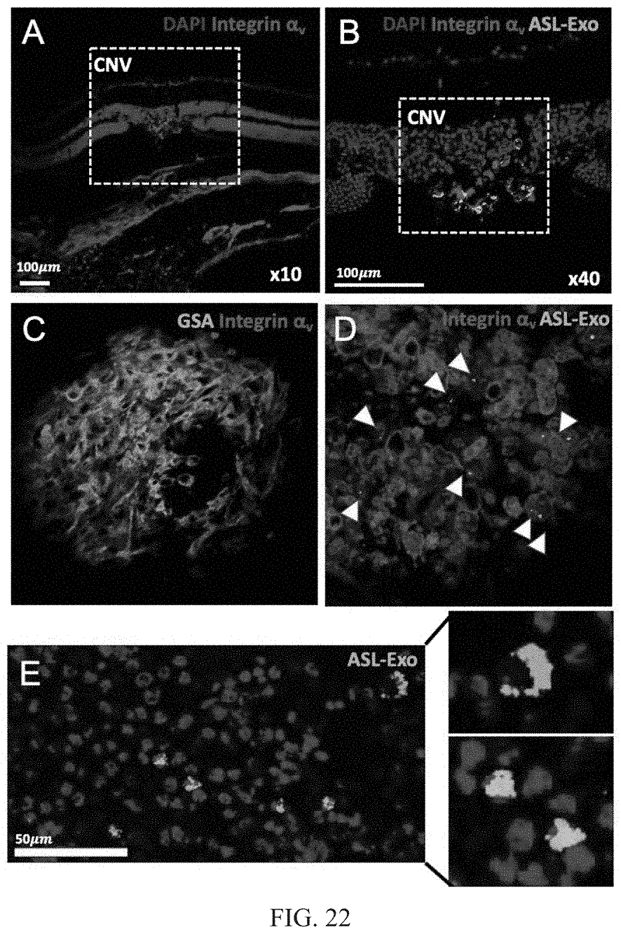

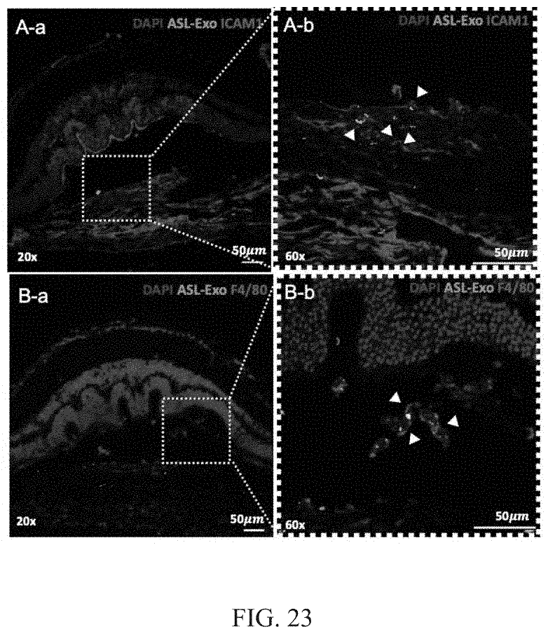

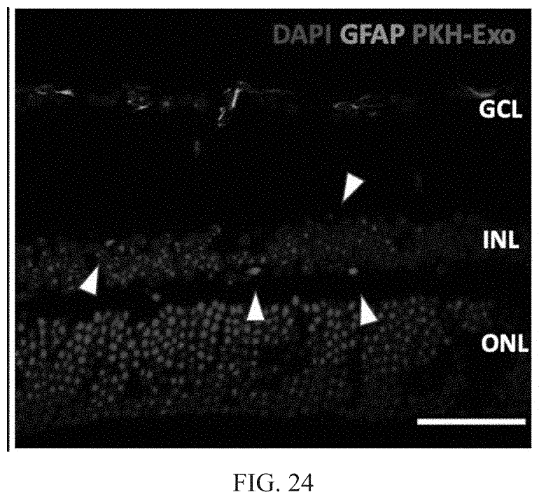

Exosomes decorated with ASL conjugates as described above were tested. A membrane anchor and spacer confer increased stability to exosomes, and RGD, one of the major integrin binding ligands, permits active targeting of integrins (FIG. 20). Of 24 different heterodimers of integrins, subsets of integrins including αVβ1, α5β1, and αIIbβ3, recognize RGD. Integrins are essential in VEGF signaling and retinal fibrosis in ocular NV. Expression of integrins αVβ3 and αVβ3 & αVβ1 is increased in ocular tissues from NVAMD and proliferative DR patients. Although RGD-mediated active targeting of integrins and angiogenesis in cancer have been utilized previously, only a few have tested active targeting of intraocular NV utilizing RGD conjugation. Previous studies reported that RGD conjugation demonstrated active targeting of choroidal NV. However, application of synthetic nanoparticles to conjugate with RGD and/or systemic administration of these particles diminished their clinical utility. Potential benefits of synthetic nanocarrier-based drug delivery, such as sustained drug delivery for the treatment of posterior eye diseases led to many fundamental studies and pre-clinical investigations of various types of synthetic nanocarriers, including polymeric nanoparticles and liposomes. Although several clinical trials (NCT03249740, NCT03953079, NCT04085341) have been and are being performed to develop nanoparticle-based treatments for posterior eye diseases, nanoparticle technology has not been clinically applied due to challenges including penetration of ocular barriers, aggregation of particles in the vitreous cavity causing visual disturbance, and immune reactions to synthetic materials in the eye. For example, to achieve various cargo deliveries to photoreceptor, RPE or choroid with efficient penetration to the retina, subretinal injections of nanocarriers were often necessary. However, subretinal injections that require an intraoperative procedure are more demanding than office-based intravitreal injections. In the present work, we used a laser-induced choroidal NV mouse model, in which Bruch's membrane and the RPE are ruptured by laser photocoagulation. This provided a model of choroidal NV in which testing of anti-VEGF agents has been predictive of outcomes in clinical trials (FIG. 21). Present results show that intravitreally-injected ASL-exosomes are delivered predominantly to choroidal NV sites colocalized with increased expressions of integrin ay. Further some of the ASL-exosomes locally delivered to choroidal NV sites were intracellular (FIG. 22). In contrast, in intravitreally-delivered naïve exosomes or ASL-exosome in the absence of laser induced CNV, retinal uptake of both exosomes were diffusedly scattered (no active targeting). Further immunohistochemistry (IHC) with anti-ICAM1 (a vascular endothelial cell marker) and anti-F4/80 (a macrophage marker) to determine the identification of these cells showed that ASL-exosomes were taken up by both vascular endothelial cells and macrophages (FIG. 23). These results support that intravitreally-delivered ASL-exosomes primarily reached areas of NV lesions. ASL-exosomes locally delivered to NV lesions both extracellularly and intracellularly, supporting that ASL-exosomes mediated intraocular drug delivery systems can be used for both extracellular and intracellular molecular targeting.

Intraocular ASL-exosome treatment was also evaluated for possible induction of reactive gliosis as a side effect. Results showed that there was no reactive gliosis observed after intravitreal exosome treatment (FIG. 24).

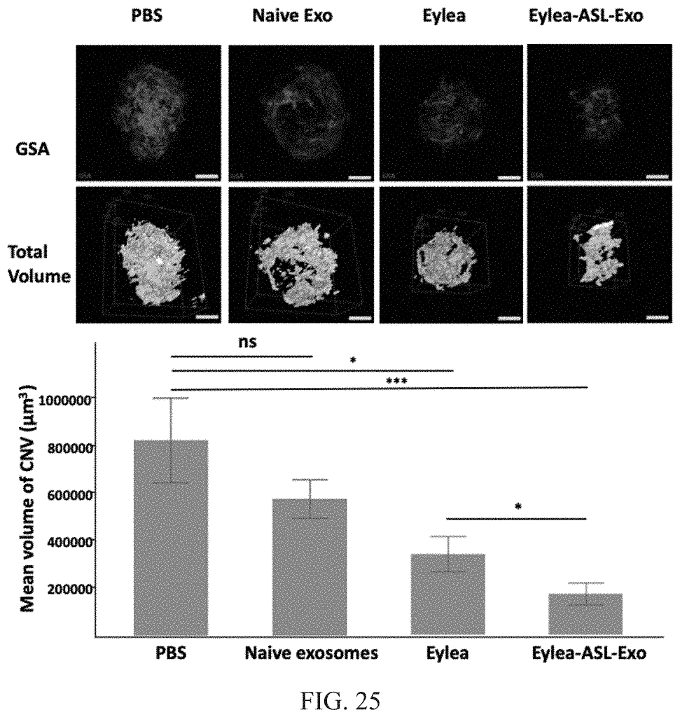

Next, we loaded ASL-exosomes with Aflibercept (Eylea®), one of the most potent anti-VEGF agents, and tested the capacity for active targeting of the loaded ASL-exosomes. Results showed that Eylea-loaded ASL-exosomes improved choroidal NV suppression in a mouse model of choroidal NV by 20% compared with Eylea-alone treatment even though ASL-exosomes contained only 10% of the amount of Eylea that was present in the group treated with Eylea alone (FIG. 25). This result demonstrates that ASL-exosome-mediated intraocular treatment allows drug loading and delivery via intravitreal injection with better efficacy by capitalizing on active targeting of NV.

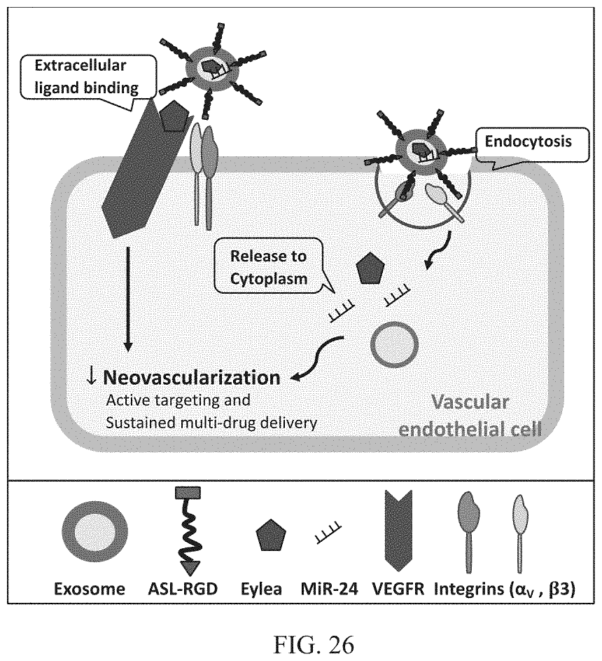

In summary, the present disclosure describes an exosome-based intraocular drug therapy with active targeting strategy using RGD modified ASL-exosomes. We demonstrated ASL-exosome can be loaded with drug and effectively deliver it to the area of retinal diseases with active targeting, and can effectively suppress ocular NV more effectively than direct intravitreal injection of drug alone. These results portend a paradigm shift in intraocular treatment from passive targeting-directed monotherapy to active exosome-based targeting-directed multi-drug delivery with sustained efficacy that can be applicable various posterior eye diseases including ocular neovascularization, infection, and inflammation (FIG. 26).

It will be understood from the foregoing description that various modifications and changes may be made in the various embodiments of the present disclosure without departing from their true spirit. The description provided herein is intended for purposes of illustration only and is not intended to be construed in a limiting sense, except where specifically indicated. Thus, while the present disclosure has been described herein in connection with certain non-limiting embodiments so that aspects thereof may be more fully understood and appreciated, it is not intended that the present disclosure be limited to these particular embodiments. On the contrary, it is intended that all alternatives, modifications, and equivalents are included within the scope of the present disclosure as defined herein. Thus the examples described above, which include particular embodiments, will serve to illustrate the practice of the present disclosure, it being understood that the particulars shown are by way of example and for purposes of illustrative discussion of particular embodiments only and are presented in the cause of providing what is believed to be a useful and readily understood description of procedures as well as of the principles and conceptual aspects of the inventive concepts. Changes may be made in the formulation of the various components, compounds, conjugates and compositions described herein, the methods described herein, or in the steps or the sequence of steps of the methods described herein, without departing from the spirit and scope of the present disclosure.

What is claimed is:

1. A modified exosome comprising:
an exosome having a surface membrane; and
a targeting modality, wherein the targeting modality comprises an anchor molecule, a spacer molecule, and a targeting ligand able to bind to a receptor, wherein the targeting ligand is conjugated to the anchor molecule by the spacer molecule; and
wherein the anchor molecule of the targeting modality is embedded within the surface membrane of the exosome such that the targeting modality extends outwardly from the surface membrane of the exosome.

2. The modified exosome of claim 1, wherein the anchor molecule is an imaging agent.

3. The modified exosome of claim 1 further comprising a therapeutic agent loaded into the exosome.

4. The modified exosome of claim 3, wherein the therapeutic agent is an anti-cancer agent.

5. The modified exosome of claim 3, wherein the therapeutic agent is an anti-infective agent.

6. The modified exosome of claim 3, wherein the therapeutic agent is an anti-neovascularization agent.

7. The modified exosome of claim 6, wherein the anti-neovascularization agent is an anti-vascular endothelial growth factor (anti-VEGF) agent.

8. The modified exosome of claim 7, wherein the anti-VEGF agent is selected from the group consisting of Bevacizumab, Ranibizumab, Aflibercept, Sorafenib, Nilotinib, Abicipar, Pazopanib, Dasatinib, Sunitinib, Telatinib, OPT-302, Faricimab, Tivozanib, Ramucirumab, Vantetanib, Regorafenib, Cabozantinib, Lenvatinib, Ponatinib, Axitinib, and Brolucizumab-dbII.

9. A method of treating a cancer in a subject in need of such therapy, comprising the step of:
administering to the subject the modified exosome of claim 1, wherein the targeting ligand binds to a receptor on the cancer, and wherein the modified exosome further comprises an anti-cancer agent loaded into the exosome.

10. A method of treating an ocular disease in a subject in need of such therapy, comprising the step of:
administering to the subject the modified exosome of claim 1, wherein the targeting ligand binds to an ocular neovascularization receptor, and wherein the modified exosome further comprises an anti-neovascularization agent loaded into the exosome.

11. The method of claim 10, wherein the anti-neovascularization agent is an anti-vascular endothelial growth factor (anti-VEGF) agent.

12. The method of claim 11, wherein the anti-VEGF agent is selected from the group consisting of Bevacizumab, Ranibizumab, Aflibercept, Sorafenib, Nilotinib, Abicipar, Pazopanib, Dasatinib, Sunitinib, Telatinib, OPT-302, Faricimab, Tivozanib, Ramucirumab, Vantetanib, Regorafenib, Cabozantinib, Lenvatinib, Ponatinib, Axitinib, and Brolucizumab-dbII.
